(19) United States
(12) Patent Application Publication
(10) Pub. No.: US 2026/0294846 A1
(43) Pub. Date: Oct. 1, 2026

(54) COMPOSITIONS AND METHODS FOR TREATING VULVAR AND VAGINAL ATROPHY (VVA)

(71) Applicant: Daré Bioscience, Inc., San Diego, CA (US)

(72) Inventor: David Friend, San Diego, CA (US)

(21) Appl. No.: 19/489,440

(22) PCT Filed: Jun. 5, 2024
(86) PCT No.: PCT/US2024/032611

§ 371 Date: Dec. 2, 2025

Related U.S. Application Data
(60) Provisional application No. 63/506,465, filed on Jun. 6, 2023.

(51) Int. Cl. A61K 31/138; A61K 9/00; A61P 15/02

(52) U.S. Cl. CPC A61K 31/138; A61K 9/0034; A61P 15/02

(57) ABSTRACT

The present disclosure generally relates to compositions and techniques for treatment of vulvar and vaginal atrophy (VVA). In some embodiments, a composition comprising tamoxifen and/or pharmaceutically acceptable salts thereof. The composition may be applied intravaginally to a vagina of a subject. In addition, certain embodiments as described herein are generally directed to techniques for making or using such compositions, kits including such compositions, or the like.

### RELATED APPLICATIONS

This application claims the benefit of priority under 35 U.S.C. § 119 (e) to U.S. Provisional Application No. 63/506,465, filed Jun. 6, 2023, the entire contents of which is incorporated herein by reference in its entirety.

### FIELD

The present disclosure generally relates to compositions and methods for treatment of vulvar and vaginal atrophy.

### BACKGROUND

Vulvar and vaginal atrophy (VVA) is a common and underreported condition that is a result of decreased estrogenization leading to thinning of the vaginal epithelium. Typical symptoms include vaginal dryness, itching, burning, and painful intercourse, adversely impacting quality of life. Clinical findings include the diagnostic presence of pale and dry vulvovaginal mucosa with petechiae, as well as a vaginal pH>5. Vulvar and vaginal atrophy most commonly occurs in the postmenopausal phase, during which the prevalence is close to 50%.

The use of estrogen-containing products for the treatment of vulvar and vaginal atrophy is contraindicated in women with hormone receptor-positive (HR+) breast cancer due to their capacity to promote tumor growth. There remains an unmet need for a novel nonhormonal VVA treatment for this subset of cancer patients and survivors, and other women who require or prefer a treatment option that does not contain estrogen.

### SUMMARY

The present disclosure generally relates to compositions and methods for treating Vulvar and vaginal atrophy (VVA) or symptom thereof, comprising inserting into a vagina a composition comprising tamoxifen. In some embodiments, the vaginal insert is a vaginal suppository. In some embodiments, the vaginal insert is a soft gelatin capsule-based vaginal insert.

The present disclosure provides a method for treating vulvar and vaginal atrophy (VVA) or one or more symptoms in a subject in need thereof, comprising administering to a vagina a composition comprising a vaginal insert and tamoxifen.

In some embodiments, the composition comprises a salt of tamoxifen. In some embodiments, the composition comprises a citrate salt of tamoxifen.

In some embodiments, the composition comprises at least about 1 mg tamoxifen. In some embodiments, the composition comprises at least about 5 mg tamoxifen. In some embodiments, the composition comprises at least about 10 mg tamoxifen. In some embodiments, the composition comprises at least about 20 mg tamoxifen.

In some embodiments, the one or more symptoms are selected from vaginal dryness, vaginal and/or vulvar irritation, vaginal and/or vulvar itching, dysuria, vaginal pain with sexual activity (dyspareunia), and/or vaginal bleeding associated with sexual activity. In some embodiments, the one or more symptoms comprises a serum level of Follicle-Stimulating Hormone (FSH) greater than 40 mIU/ml. In some embodiments, the one or more symptoms comprises a vaginal pH greater than 5. In some embodiments, the one or more symptoms comprises less than 5 percent superficial cells on a vaginal smear.

In some embodiments, the subject is a postmenopausal woman with a uterus or a postmenopausal woman without a uterus. In some embodiments, the subject is a hormone adverse woman. In some embodiments, the subject is a woman with current or a history of breast cancer. In some embodiments, the subject is a woman with current or a history of hormone receptor-positive (HR+) breast cancer. In some embodiments, the subject is a woman with a history of breast cancer or a mammogram that is positive or suspect for breast cancer or breast cancer occurring in an identical twin. In some embodiments, the subject is a woman with a history of thrombophilia.

In some embodiments, the composition is administered to a vagina by self-placement. In some embodiments, the composition is administered one dose per day for 14 days followed by two doses per week for 6 weeks. In some embodiments, the method comprises measurement of serum levels of 4-hydroxytamoxifen, N-desmethyltamoxifen, and/or N-desmethyl-4-hydroxytamoxifen. In some embodiments, the method comprises measurement of vaginal pH. In some embodiments, the method comprises measurement Vaginal Maturation Index (VMI).

The present disclosure provides a pharmaceutical composition comprising a vaginal insert and tamoxifen. In some embodiments, the vaginal insert is a vaginal suppository. In some embodiments, the vaginal insert is a soft gelatin capsule-based vaginal insert. In some embodiments, the vaginal insert is a vaginal tablet. In some embodiments, the vaginal insert is a soft gelatin capsule. In some embodiments, the composition comprises at least about 1 mg tamoxifen. In some embodiments, the composition comprises at least about 5 mg tamoxifen. In some embodiments, the composition comprises at least about 10 mg tamoxifen. In some embodiments, the composition comprises at least about 20 mg tamoxifen.

The present disclosure provides a kit comprising the pharmaceutical composition of the present disclosure and a dispenser for the composition. In some embodiments, the dispenser contains a single unit dosage form of the composition. In some embodiments, a single unit dosage comprises between about 1 mg and about 20 mg of the composition. In some embodiments, the kit comprises multiple dispensers and each dispenser contains a single unit dosage of the composition.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

FIG. 1 is a diagram of a Study Design for a phase 1/2, randomized, multi-center, double-blind, parallel-arm, placebo-controlled, dose-ranging study to evaluate safety, tolerability, pharmacokinetics (PK), and pharmacodynamics (PD) of a tamoxifen vaginal insert.

FIG. 2 is a schematic of the disposition of participants. IP—investigational product; ITT—intent to treat; and PK—pharmacokinetic.

FIGS. 3A-3L are a series of graphs depicting systemic plasma pharmacokinetics. FIG. 3A Plasma tamoxifen; 1 mg dosing group. FIG. 3B Plasma tamoxifen; 5 mg dosing group. FIG. 3C Plasma tamoxifen; 10 mg dosing group. FIG. 3D Plasma tamoxifen; 20 mg dosing group. FIG. 3E Plasma NDT; 1 mg dosing group. FIG. 3F Plasma NDT; 5 mg dosing group. FIG. 3G Plasma NDT; 10 mg dosing group. FIG. 3H Plasma NDT; 20 mg dosing group. FIG. 3I Plasma endoxifen; 10 mg dosing group. FIG. 3J Plasma endoxifen; 20 mg dosing group. FIG. 3K Plasma 4-OHT; 10 mg dosing group. FIG. 3L Plasma 4-OHT; 20 mg dosing group.

EOT—end of treatment; LLOQ—lower limit of quantitation; NDT—N-desmethyltamoxifen; 4-OHT—4-hydroxytamoxifen.

### DETAILED DESCRIPTION

Vulvar and vaginal atrophy (VVA) is a condition that is a result of decreased estrogenization leading to thinning of the vaginal epithelium. Clinical findings include the diagnostic presence of pale and dry vulvovaginal mucosa with petechiae, as well as a vaginal pH>5. Vulvar and vaginal atrophy most commonly occurs in the postmenopausal phase.

Over-the-counter (OTC) remedies such as vaginal moisturizers and lubricants can provide temporary symptomatic treatment at the time of use. Localized estrogen therapy (i.e., estrogen-containing creams, rings, vaginal tablets, vaginal inserts, and suppositories) can treat the underlying physiologic issue and is the most commonly prescribed treatment by physicians for VVA. However, this therapeutic approach is contraindicated for women with hormone receptor-positive (HR+) breast cancer. In addition to surgery and/or radiation, many of these women are treated with aromatase inhibitors (AIs), which deplete the body of estrogen and induce a menopausal state irrespective of age. Consequently, the prevalence of VVA in HR+ breast cancer survivors is an estimated 70%.

Although localized estrogen therapy is highly effective in reducing the symptoms of VVA in otherwise healthy women, it is a suboptimal intervention for HR+ patients and survivors because of the prolific response of these tumors to the presence of estrogen in the surrounding tissue. As a result of this safety concern, the current estrogen products on the market for the treatment of VVA are labeled as being contraindicated when the patient has known breast cancer, suspected breast cancer, or a history of breast cancer. Oncologists, obstetricians, gynecologists, and primary care providers are reluctant to prescribe any estrogen-based products owing to concerns of reintroducing estrogen into the system, despite the low dose and topical delivery.

The present disclosure generally relates to compositions and methods for local application of tamoxifen to mitigate the symptoms of VVA for patients with or at risk for HR+ breast cancer, including women currently on anticancer therapy. There remains a large unmet need for a nonhormonal VVA treatment specifically developed for this subset of cancer patients and survivors, and other women who require or prefer a treatment option that does not contain estrogen.

Selective estrogen receptor modulators (SERMs) represent an option for the treatment of VVA in an HR+ patient population. Tamoxifen is a well-characterized SERM that has been prescribed by oncologists for the treatment of breast cancer. Tamoxifen acts as an estrogen antagonist in the breast; it binds estrogen receptors in the breast tissue, effectively blocking natural estrogen from binding to these sites, and is therefore an effective HR+ breast cancer treatment. In contrast, in other tissues such as uterine and vaginal tissues, tamoxifen acts as an estrogen agonist, producing similar physiological responses to those produced by estrogen, without being a true estrogen.

Localized tamoxifen therapy has the potential to counter the physiologic changes that lead to VVA without introducing estrogen back into the system. The present disclosure provides a tamoxifen vaginal insert which unexpectedly offers several potential clinical advantages over oral administration of tamoxifen, including: direct delivery to the site of action at therapeutic levels while reducing systemic exposure, thereby reducing known adverse events. The tamoxifen vaginal insert of the present disclosure is provided for use in the treatment of postmenopausal women with VVA, including women with a history of breast cancer.

### Composition

### SERM

Selective estrogen receptor modulators (SERMs) and/or its analog, derivative, isomer, metabolite, pharmaceutically acceptable salt, pharmaceutical product, hydrate, N-oxide, or any combination thereof may be used in the compositions and methods of the present disclosure. The term selective estrogen receptor modulator includes both estrogen agonist and estrogen antagonists and refers to compounds that bind with the estrogen receptor, inhibit bone turnover and prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues.

SERM-like activity can be classified within four groups by chemical structure: triphenylethylene compounds, benzopyrans, naphthalenes, and benzothiophenes. SERMs encompassed by the present invention include, but are not limited to the following triphenylalkylenes such as triphenylethylenes, which include tamoxifen and associated compounds which are disclosed in U.S. Pat. No. 4,536,516, the disclosure of which is hereby incorporated by reference; 4-hydroxy tamoxifen which is disclosed in U.S. Pat. No. 4,623,660, the disclosure of which is hereby incorporated by reference; toremifine, droloxifene, yoremifene, idoxifene (Pyrrolidine, 1-1-[4-[-1-(4-iodophenyl)-2-phenyl-1-Butenyl]phenoxy]ethyl]) and associated compounds which are disclosed in U.S. Pat. No. 4,839,155, the disclosure of which is hereby incorporated by reference; clomiphene, enclomiphene and zuclomiphene; benzothiphene derivatives such as raloxifene (methanone, [6-hydroxy-2-(4-hydroxyphenyl)benzo[b]thien-3-yl][4-[2-(1-piperidinyl) ethoxy]phenyl]-, hydrochloride) and associated compounds which are disclosed in U.S. Pat. No. 4,418,068, the disclosure of which is hereby incorporated by reference; and LY 353381; benzopyran derivatives such as EM 800 (SCH 57050) and its metabolite EM 652; naphthalene derivatives such as lasofoxifene (CP 336,156); chromans such as levormeloxifene or their analogs, derivatives, isomers, or metabolites thereof, or their pharmaceutically acceptable salts, esters, N-oxides, or mixtures thereof. Preferably, the triphenylethylene compounds are used in the present disclosure.

### Tamoxifen

Tamoxifen is a selective estrogen receptor modulator. The chemical name is 1-(p-dimethylamino-ethoxphenyl)-2-ethyl-1,2-diphenylethylene. Tamoxifen has an empirical formula of C₂₆H₂₉NO, and has a molecular weight of 371.521. In the present disclosure, both the cis and trans isomers are contemplated.

In some embodiments, tamoxifen is in the form of tamoxifen citrate. The chemical name of tamoxifen citrate is (Z) 2-[4-(1,2-diphenyl-1-butenyl) phenoxy]-N, N-dimethylethanamine 2-hydroxy-1,2,3-propanetricarboxylate. Tamoxifen citrate has an empirical formula of C₂₆H₂₉NO·C₆H₈O₇ and has a molecular weight of 563.62. The structure is shown as Formula I:

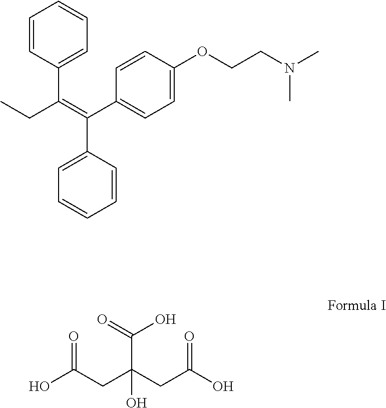 [SMILES: CC/C(=C(\c1ccccc1)c1ccc(OCCN(C)C)cc1)c1ccccc1 ; O=C(O)CC(O)(CC(=O)O)C(=O)O]

In some embodiments, tamoxifen is present in about 0.2 mg per dose. In some embodiments, tamoxifen is present in about 0.5 mg per dose. In some embodiments, tamoxifen is present in about 1 mg per dose. In some embodiments, tamoxifen is present in about 1.5 mg per dose. In some embodiments, tamoxifen is present in about 2 mg per dose. In some embodiments, tamoxifen is present in about 2.5 mg per dose. In some embodiments, tamoxifen is present in about 3 mg per dose. In some embodiments, tamoxifen is present in about 4 mg per dose. In some embodiments, tamoxifen is present in about 5 mg per dose. In some embodiments, tamoxifen is present in about 6 mg per dose. In some embodiments, tamoxifen is present in about 7 mg per dose. In some embodiments, tamoxifen is present in about 8 mg per dose. In some embodiments, tamoxifen is present in about 9 mg per dose. In some embodiments, tamoxifen is present in about 10 mg per dose. In some embodiments, tamoxifen is present in about 11 mg per dose. In some embodiments, tamoxifen is present in about 12 mg per dose. In some embodiments, tamoxifen is present in about 13 mg per dose. In some embodiments, tamoxifen is present in about 14 mg per dose. In some embodiments, tamoxifen is present in about 15 mg per dose. In some embodiments, tamoxifen is present in about 16 mg per dose. In some embodiments, tamoxifen is present in about 17 mg per dose. In some embodiments, tamoxifen is present in about 18 mg per dose. In some embodiments, tamoxifen is present in about 19 mg per dose. In some embodiments, tamoxifen is present in about 20 mg per dose. In some embodiments, tamoxifen is present in about 21 mg per dose. In some embodiments, tamoxifen is present in about 22 mg per dose. In some embodiments, tamoxifen is present in about 23 mg per dose. In some embodiments, tamoxifen is present in about 24 mg per dose. In some embodiments, tamoxifen is present in about 25 mg per dose.

In some embodiments, tamoxifen is present in amounts ranging from about 0.2 mg per dose to about 10 mg per dose. In some embodiments, tamoxifen is present in amounts ranging from about 1 mg per dose to about 12 mg per dose. In some embodiments, tamoxifen is present in amounts ranging from about 4 mg per dose to about 15 mg per dose. In some embodiments, tamoxifen is present in amounts ranging from about 8 mg per dose to about 17 mg per dose. In some embodiments, tamoxifen is present in amounts ranging from about 10 mg per dose to about 20 mg per dose. In some embodiments, tamoxifen is present in amounts ranging from about 14 mg per dose to about 25 mg per dose.

In some embodiments, tamoxifen is present in a suppository dosage form. In some embodiments, tamoxifen is present as tamoxifen citrate. In some embodiments, tamoxifen is present in a vaginal tablet dosage form. In some embodiments, tamoxifen is present in a soft gelatin capsule-based vaginal insert dosage form.

In some embodiments, tamoxifen is formulated into a pharmaceutical composition with additional constituents for vaginal administration by way of suppositories, creams, foams, gels (including, but not limiting to aqueous solutions and suspensions), ointments, tablets, ovules, pessaries and rings, and other known pharmaceutically acceptable carriers known in the art.

In one embodiment, tamoxifen is formulated with a fatty base. The base may be selected from, but is not limited to JAB base, JC base, polyethylene glycol base, emollient cream, vanishing cream light, vanpen base, cosmetic HRT base, or mixtures thereof. When the mode of administration is through a vaginal suppository, the base may be JAB. JAB base is a combined formulation comprising Base K, Base C and Base M. Base K is composed of PEG-8 distearate. Base C is composed of a hydrogenated vitamin oil. Base M is composed of Vitamin E Acetate.

The pharmaceutical composition may include one or more additives, depending on the pharmaceutically acceptable carrier, a preservative, a dye, a binder, a suspending agent, a dispersing agent, a colorant, a disintegrate, an excipient, a diluent, a lubricant, a plasticizer, an oil or any combination of any of the foregoing. In some embodiments, silica gel is used as a suspending agent.

The pharmaceutical composition may include one or more of Hard Fat (Wecobee® FS), Mineral Oil, and White Petrolatum. In some embodiments, the formulation comprises Hard Fat (Wecobee® FS), Mineral Oil, and White Petrolatum. In some embodiments, the formulation consists of Hard Fat (Wecobee@ FS), Mineral Oil, and White Petrolatum.

Suitable pharmaceutically acceptable additives include, but are not limited to, ethanol; water; glycerol; aloe vera gel; allantoin; glycerin; vitamin A and E oils; mineral oil; PPG2 myristyl propionate; vegetable oils and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; polyethylene glycol; waxes; and the like.

Suitable lubricants include, but are not limited to, sodium oleate, sodium stearate, magnesium stearate, sodium acetate, and the like.

The composition may also include suitable preservatives, e.g., sodium benzoate, and other additives that may render the composition more suitable for application, e.g., sodium chloride, which affects the osmolarity of the preparation.

Suitable dispersing and suspending agents include, but are not limited to synthetic and natural gums, such as bentoite, vegetable gum, tragacanth, acacia, alginate, dextran, sodium carboxymethylcellulose, methylcellulose, polyvinyl-pyrrolidone and gelatin.

A suitable pharmaceutical diluent is, but is not limited to, water.

Suitable lubricants include, but are not limited to, sodium oleate, sodium stearate, magnesium stearate, sodium acetate, and the like.

Additionally, various agents may be used to change the pH of the composition as necessary, including, for example, hydrochloric acid or sodium hydroxide, and antioxidants such as citric acid, ascorbic acid, fumaric acid and malic acid. Other illustrative antioxidants include palmitate, butylated hydroxyanisole, propylgallate, sodium ascorbate, and sodium metabisulfite.

In embodiments, the tamoxifen and/or composition is as described in U.S. Pat. No. 9,480,662, which is incorporated herein by reference in its entirety.

### Delivery

Many methods may be used for vaginal administration of the formulation of the present disclosure. These include vaginal administration of creams, suppositories, foams, gels (including, but not limited to, aqueous solutions and suspensions) ointments, tablets, ovules, pessaries and rings. In some embodiments, the compositions of the present disclosure are administered as a vaginal suppository.

In some embodiments, the triphenylethylene derivatives may be formulated together or separately. The composition may be formulated per unit dose, or labeled for dispensing an amount, such that each dosage contains from about 0.2 mg to about 25 mg per unit dose of tamoxifen.

The pharmaceutical composition may be in a “unit dosage form”, which refers to physically discrete units suitable as unitary dosages for human subjects and other mammals, each unit containing a predetermined quantity of active material calculated to produce the desired therapeutic effect, in association with one or more of the above-described suitable pharmaceutical diluents, excipients or carriers.

In an embodiment, tamoxifen is formulated into a tablet for delivery to the vaginal tissue. For example, a tablet is inserted in an applicator and delivered to the vaginal area. The tablet is designed to have adhesive ability once contacted with vaginal moisture. The tablet is designed to slowly dissolve over a number of hours. The advantages of such a delivery method over gels, creams and ointments include reduced messiness and limited leakage from the vaginal area of the active substance tamoxifen.

For example, pharmaceutical preparations for tablet vaginal administration can be obtained by combining the sole active agent tamoxifen with solid carriers, where appropriate granulating a resulting mixture, and processing the mixture or granulate, if desired or necessary after the addition of suitable adjuncts, into tablets or dosage cores. Suitable carriers are sugars, for example, lactose, saccharose, mannitol or sorbitol, cellulose preparations and/or calcium phosphates, for example, tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, cross-linked polyvinylpyrrolidone, agar or alginic acid or a salt thereof, such as sodium alginate. Adjuncts are especially flow-regulating agents and lubricants, for example, silicic acid, talc, stearic acid or salts thereof, such as magnesium or calcium stearate, and/or polyethylene glycol. The cores may be enclosed in suitable coatings such as concentrated sugar solutions that optionally contain gum arabic, polyvinylpyrrolidone, polyethylene glycol, suitable cellulose preparations, such as acetylcellulose phthalate or hydroxypropylmethylcellulose. Coloring substances or pigments may be added to the tablets or drage coatings, for example for the purpose of identification or to indicate different doses of active ingredient.

In another aspect, the present disclosure is directed to a kit including one or more of the compositions discussed herein. A “kit,” as used herein, refers to a package or an assembly including one or more of the compositions of the disclosure, and/or other compositions associated with the disclosure, for example, as described herein. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the disclosure include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the disclosure may, in some cases, include instructions in any form that are provided in connection with the compositions of the disclosure in such a manner that one of ordinary skill in the art would recognize that the instructions are to be associated with the compositions of the disclosure. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject.

### Methods of Use

The present disclosure provides a method, comprising: applying, to a vagina of a subject, a composition comprising an active ingredient for treating VVA. In some embodiments, the active ingredient is tamoxifen. In some embodiments, the subject has VVA. In some embodiments, the subject is at risk of VVA. In some embodiments, the composition is a vaginal insert. In some embodiments, the vaginal insert is a vaginal suppository. In some embodiments, the active ingredient is present at about 1-20 mg. In some embodiments, the composition is administered to a vagina and/or intravaginally. In some embodiments, the composition is applied as a single dose. In some embodiments, the composition is applied as at least one dose.

The active ingredient may include tamoxifen, as well as salts thereof. Compositions and methods for applying the compositions of the present disclosure for the treatment or prevention of indications such as any of those described herein, e.g., to the vagina of a subject, such as a human. The subject can also be a non-human animal. In embodiments, the subject is female. In embodiments, the subject is a woman.

A composition such as described herein may be used to treat a subject having or at risk of moderate to severe symptoms of vulvar and vaginal atrophy (VVA) in hormone adverse women. The term “hormone adverse women” as used herein includes women with current or a history of hormone receptor positive (HR+) breast cancer. Hormone adverse women includes women who are currently on anticancer therapy or women for whom estrogen therapy is not recommended, for example, those with a history of thrombophilia. In another set of embodiments, a composition such as described herein may be used to treat a subject having or at risk of VVA. In embodiments, the composition is administered to a subject at least one time.

In some embodiments, symptoms of VVA include, but are not limited to, vaginal dryness, vaginal and/or vulvar irritation/itching, dysuria, vaginal pain with sexual activity (dyspareunia), the presence of vaginal bleeding associated with sexual activity, and any combination thereof. In some embodiments, compositions of the present disclosure are administered intravaginally.

Compositions disclosed herein may comprise a tamoxifen vaginal suppository. In some embodiments, about 0.5 grams of tamoxifen is administered to the subject. In some embodiments, about 1 grams of tamoxifen is administered to the subject. In some embodiments, about 2 grams of tamoxifen is administered to the subject. In some embodiments, about 3 grams of tamoxifen is administered to the subject. In some embodiments, about 4 grams of tamoxifen is administered to the subject. In some embodiments, about 5 grams of tamoxifen is administered to the subject. In some embodiments, about 6 grams of tamoxifen is administered to the subject. In some embodiments, about 7 grams of tamoxifen is administered to the subject. In some embodiments, about 8 grams of tamoxifen is administered to the subject. In some embodiments, about 9 grams of tamoxifen is administered to the subject. In some embodiments, about 10 grams of tamoxifen is administered to the subject. In some embodiments, about 12 grams of tamoxifen is administered to the subject. In some embodiments, about 14 grams of tamoxifen is administered to the subject. In some embodiments, about 15 grams of tamoxifen is administered to the subject. In some embodiments, about 16 grams of tamoxifen is administered to the subject. In some embodiments, about 18 grams of tamoxifen is administered to the subject. In some embodiments, about 20 grams of tamoxifen is administered to the subject. In some embodiments, about 22 grams of tamoxifen is administered to the subject. In some embodiments, about 24 grams of tamoxifen is administered to the subject. In some embodiments, about 25 grams of tamoxifen is administered to the subject.

In some embodiments, about 0.5 grams to about 5 grams of the tamoxifen is administered to the subject. In some embodiments, about 1 gram to about 6 grams of the tamoxifen is administered to the subject. In some embodiments, about 2 grams to about 8 grams of the tamoxifen is administered to the subject. In some embodiments, about 5 grams to about 10 grams of the tamoxifen is administered to the subject. In some embodiments, about 6 grams to about 12 grams of the tamoxifen is administered to the subject. In some embodiments, about 10 grams to about 15 grams of the tamoxifen is administered to the subject. In some embodiments, about 12 grams to about 18 grams of the tamoxifen is administered to the subject. In some embodiments, about 15 grams to about 20 grams of the tamoxifen is administered to the subject. In some embodiments, about 17 grams to about 23 grams of the tamoxifen is administered to the subject. In some embodiments, about 20 grams to about 25 grams of the tamoxifen is administered to the subject.

In some embodiments, the single unit dosage of tamoxifen vaginal insert is about 1 gram. In some embodiments, the single unit dosage of tamoxifen vaginal insert is about 2 grams. In some embodiments, the single unit dosage of tamoxifen vaginal insert is about 3 gram. In some embodiments, the single unit dosage of tamoxifen vaginal insert is about 4 grams. In some embodiments, the single unit dosage of tamoxifen vaginal insert is about 5 gram. In some embodiments, the single unit dosage of tamoxifen vaginal insert is about 7 grams. In some embodiments, the single unit dosage of tamoxifen vaginal insert is about 9 gram. In some embodiments, the single unit dosage of tamoxifen vaginal insert is about 10 grams. In some embodiments, the single unit dosage of tamoxifen vaginal insert is about 12 gram. In some embodiments, the single unit dosage of tamoxifen vaginal insert is about 14 grams. In some embodiments, the single unit dosage of tamoxifen vaginal insert is about 15 gram. In some embodiments, the single unit dosage of tamoxifen vaginal insert is about 17 grams. In some embodiments, the single unit dosage of tamoxifen vaginal insert is about 19 gram. In some embodiments, the single unit dosage of tamoxifen vaginal insert is about 20 grams. In some embodiments, the single unit dosage of tamoxifen vaginal insert is about 22 gram. In some embodiments, the single unit dosage of tamoxifen vaginal insert is about 24 grams. In some embodiments, the single unit dosage of tamoxifen vaginal insert is about 25 gram. In some embodiments, the single unit dosage of tamoxifen vaginal insert is about 27 grams. In some embodiments, the single unit dosage of tamoxifen vaginal insert is about 29 gram. In some embodiments, the single unit dosage of tamoxifen vaginal insert is about 30 grams.

In some embodiments, the single unit dosage of the composition is applied intravaginally. In some embodiments, the tamoxifen vaginal insert is not inserted within 1 cm the cervix. In some embodiments, the composition is applied intravaginally to the anterior distal vagina at a depth of between about 0 cm and about 3 cm in the vagina. In some embodiments, the composition is applied intravaginally to the anterior distal vagina at a depth of between about 0 cm and about 1 cm. In some embodiments, the composition is applied intravaginally to the anterior distal vagina at a depth of between about 0 cm and about 1.5 cm. In some embodiments, the composition is applied intravaginally to the anterior distal vagina at a depth of between about 0 cm and about 2 cm. In some embodiments, the composition is applied intravaginally to the anterior distal vagina at a depth of between about 0 cm and about 2.5 cm. In some embodiments, the composition is applied intravaginally to the anterior distal vagina at a depth of between about 0 cm and about 3 cm. In some embodiments, the composition is applied intravaginally to the anterior distal vagina at a depth of between about 0 cm and about 4 cm.

In some embodiments, a first dose of the composition is administered at least about 1 day before a second dose of the composition. In some embodiments, a first dose of the composition is administered at least about 2 days before a second dose of the composition. In some embodiments, a first dose of the composition is administered at least about 3 days before a second dose of the composition. In some embodiments, a first dose of the composition is administered at least about 4 days before a second dose of the composition. In some embodiments, a first dose of the composition is administered at least about 5 days before a second dose of the composition. In some embodiments, a first dose of the composition is administered at least about 6 days before a second dose of the composition. In some embodiments, a first dose of the composition is administered at least about 7 days before a second dose of the composition.

In some embodiments, a unit dose of the composition is administered once per day for 7 consecutive days. In some embodiments, a unit dose of the composition is administered once per day for 8 consecutive days. In some embodiments, a unit dose of the composition is administered once per day for 9 consecutive days. In some embodiments, a unit dose of the composition is administered once per day for 10 consecutive days. In some embodiments, a unit dose of the composition is administered once per day for 11 consecutive days. In some embodiments, a unit dose of the composition is administered once per day for 12 consecutive days. In some embodiments, a unit dose of the composition is administered once per day for 13 consecutive days. In some embodiments, a unit dose of the composition is administered once per day for 14 consecutive days. In some embodiments, a unit dose of the composition is administered once per day for 15 consecutive days.

In some embodiments, a unit dose of the composition is administered twice per week for 2 consecutive weeks. In some embodiments, a unit dose of the composition is administered twice per week for 3 consecutive weeks. In some embodiments, a unit dose of the composition is administered twice per week for 4 consecutive weeks. In some embodiments, a unit dose of the composition is administered twice per week for 5 consecutive weeks. In some embodiments, a unit dose of the composition is administered twice per week for 6 consecutive weeks. In some embodiments, a unit dose of the composition is administered twice per week for 7 consecutive weeks. In some embodiments, a unit dose of the composition is administered twice per week for 8 consecutive weeks. In some embodiments, a unit dose of the composition is administered twice per week for 9 consecutive weeks. In some embodiments, a unit dose of the composition is administered twice per week for 10 consecutive weeks.

In some embodiments, a female subject exhibiting one or more symptom of VVA has not been administered at least one of anticoagulant drugs, Class 1 antiarrhythmics, or digitalis within about 12 months prior to the administration of a first dose of the composition.

In some embodiments, a female subject exhibiting one or more symptom of VVA has not been administered at least one vaginal hormonal product within about 28 days prior to the administration of a first dose of the composition. In some embodiments, a female subject exhibiting one or more symptom of VVA has not been administered at least one transdermal estrogen product within about 28 days prior to the administration of a first dose of the composition. In some embodiments, a female subject exhibiting one or more symptom of VVA has not been administered at least one oral estrogen therapy within about 56 days prior to the administration of a first dose of the composition. In some embodiments, a female subject exhibiting one or more symptom of VVA has not been administered at least one estrogen-alone injectable drug therapy within about 84 days prior to the administration of a first dose of the composition. In some embodiments, a female subject exhibiting one or more symptom of VVA has not been administered at least one estrogen pellet therapy within about 6 months prior to the administration of a first dose of the composition. In some embodiments, a female subject exhibiting one or more symptom of VVA has not been administered at least one of SERMs or aromatase inhibitors (AIs) within about 6 months prior to the administration of a first dose of the composition. In some embodiments, a female subject exhibiting one or more symptom of VVA has not been administered at least one anabolic or other steroid product (including hormonal creams such as testosterone) within about 28 days prior to the administration of a first dose of the composition. In some embodiments, a female subject exhibiting one or more symptom of VVA has not been administered corticosteroids, >5 mg/day prednisone or equivalent for more than 4 weeks within about 28 days prior to the administration of a first dose of the composition. In some embodiments, a female subject exhibiting one or more symptom of VVA has not been administered at least one of intravaginal products (e.g., Femring® [estradiol acetate vaginal ring], ESTRING® [estradiol vaginal ring]) within about 21 days prior to the administration of a first dose of the composition. In some embodiments, a female subject exhibiting one or more symptom of VVA has not consumed St John's Wort (hypericum perforatum), grapefruit, Seville (bitter) orange, or pomelo fruit/juice/preserves within about 21 days prior to the administration of a first dose of the composition.

In some embodiments, a female subject exhibiting one or more symptom of VVA has not been administered at least one of the following CYP3A4/5 inducers, including, but not limited to: Apalutamide, aprepitant, armodafinil, Bosentan, Carbamazepine, corticosteroids, Dabrafenib, Efavirenz, encorafenib, enzalutamide, etravirine, Loriatinib, lumacaftor, Modafinil, Nevirapine Phenobarbital, phenytoin, Rifabutin, rifampicin, ritonavir, rufinamide, Tipranavir, or Vemurafenib within about 21 days prior to the administration of a first dose of the composition.

In some embodiments, a female subject exhibiting one or more symptom of VVA has not been administered at least one of the following CYP2D6 inhibitors, including, but not limited to: Abiraterone, amiodarone, Bupropion, Celecoxib, cinacalcet, cobicistat, Duloxetine, Fluoxetine, Methadone, mirabegron, Paroxetine, or Terbinafine within about 21 days prior to the administration of a first dose of the composition. In some embodiments, a female subject exhibiting one or more symptom of VVA has not been administered vaginal lubricants within 6 hours of administration of the tamoxifen vaginal insert.

In embodiments, the subject does not have history of significant cardiovascular, renal, pulmonary, neurological and hepatic diseases. In embodiments, the subject does not have history of coagulopathies, thrombophilia or thromboembolic disease (deep vein thrombosis, pulmonary or systemic embolism, stroke, or transient ischemic attack). In embodiments, the subject does not have history of uncontrolled hypertension (either systolic>180 mmHg or diastolic>105 mmHg). In embodiments, the subject does not have history of congestive heart failure (New York Heart Association [NYHA]>Class I), or myocardial infarction within 12 months prior to the administration of a first dose of the composition. In embodiments, the subject does not have history of abnormal cervical screening test within 2 years of screening. In embodiments, the subject does not have history of endometrial pathology: hyperplasia, carcinoma and/or polyp in subjects who have not undergone hysterectomy. In embodiments, the subject does not have history of breast cancer within 5 years of screening. In embodiments, if the subject has a history of breast cancer more than 5 years prior to screening, their disease was node-negative, nonmetastatic, and all treatment with aromatase inhibitors (AIs) or SERMs was completed at least 6 months prior to screening. In embodiments, the subject does not have history of malignant melanoma. In embodiments, the subject does not have history of any cancer (except nonmelanomatous skin cancer) diagnosed less than 5 years prior to the administration of a first dose of the composition. In embodiments, the subject does not have history of undiagnosed vaginal bleeding. In embodiments, the subject does not have history of known or suspected estrogen-dependent neoplasia. In embodiments, the subject does not have history of radiation treatment to the pelvis. In embodiments, the subject does not have history of unsatisfactory outcome from a vaginal hormone therapy for VVA. In embodiments, the subject does not have history of hypersensitivity to tamoxifen. In embodiments, the subject does not have history of clinically significant uterine fibroids in subjects who have not undergone hysterectomy.

### Definitions

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

Terms such as “treat,” “treatment,” “treating,” etc. comprise therapeutic treatment of subjects having already developed a disease or disorder, in particular in manifest form. Therapeutic treatment may be symptomatic treatment in order to relieve the signs and/or symptoms of the disease or disorder or causal treatment in order to reverse, partially reverse, stop, or slow down the progression of the disease or disorder. Thus, the compositions and methods of the present disclosure may be used, for instance, as therapeutic treatment (e.g., for acute or chronic therapy).

Additionally, terms such as “prevent,” “preventing,” or “prevention” generally refer to the reduction of the occurrence of the disease or disorder, and/or a sign and/or symptom thereof, in the treated sample relative to an untreated control sample, or delays the onset of one or more signs and/or symptoms of the disease or disorder relative to the untreated control sample, in a statistically significant manner. Preventing the disease or disorder, and/or a sign and/or a symptom thereof, includes preventing or delaying the initiation of the disease, disorder, sign, and/or symptom. Prevention also includes preventing a recurrence of the disease, disorder, sign, and/or symptom.

In certain aspects, the composition can be applied to a subject, e.g., to the vagina of a subject, and/or to another body cavity, for example, the mouth or the rectum. Any suitable technique may be used to apply the composition to the subject. For instance, the composition may be free or mass flowing, e.g., so that it may be administered through an applicator or other suitable device. Thus, in some embodiments, the composition may be contained within applicator, such as a vaginal applicator or a syringe, which can be applied, e.g., by the subject, or by another person.

The subject may be, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., monkeys such as cynomolgus monkeys or rhesus monkeys, chimpanzees, etc.); commercially relevant mammals such as cattle, pigs, horses, sheep, rabbits, mice, rats, goats, cats, dogs, etc.) and birds (e.g., commercially relevant birds such as chickens, ducks, geese, turkeys, etc.). In certain embodiments, the subject is a mammal. The subject may be a female and at any stage of development. A non-human animal may be a transgenic animal.

In one set of embodiments, as discussed, the composition is applied to treat the subject with a therapeutically effective amount of an active ingredient, such as any of those described herein. The therapeutically effective amount may be an amount which, when administered to a subject for treating or preventing a disease or disorder, is sufficient to effect such treatment or prevention for the disease or disorder, for example, any of those described herein. The therapeutically effective amount may also be an amount sufficient to elicit a desired biological response, i.e., alleviating a symptom. The therapeutically effective amount may vary depending on such factors as the desired biological endpoint, the mode of administration, and/or the age and health of the subject.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%. When the word “about” is used herein in reference to a number, it should be understood that still another embodiment of the disclosure includes that number not modified by the presence of the word “about.”

When ranges are given by specifying the lower end of a range separately from the upper end of the range, it will be understood that the range can be defined by selectively combining any one of the lower end variables with any one of the upper end variables that is mathematically possible. Where ranges are recited, it will be understood that any subrange or value within the recited ranges, including endpoints, is contemplated.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### LIST OF ABBREVIATIONS

| Abbreviation | Explanation |
|---|---|
| λz | terminal rate constant |
| ADR | adverse drug reaction |
| AE | adverse event |
| AI | aromatase inhibitor |
| APTT | activated partial thromboplastin time |
| ATC | Anatomical Therapeutic Chemical |
| AUC | area under the plasma concentration-time curve |
| BMI | body mass index |
| BP | blood pressure |
| Cₘₐₓ | maximum observed plasma drug concentration |
| CRA | clinical research associate |
| CRF | case report form |
| CSR | clinical study report |
| CST | cervical screening test |
| DMP | data management plan |
| DVT | deep vein thrombosis |
| ECG | electrocardiogram |
| EDC | electronic data capture |
| eCRF | electronic case report form |
| FSH | follicle-stimulating hormone |
| FU | follow-up |
| GCP | Good Clinical Practice |
| HBsAg | hepatitis B surface antigen |
| HCV | hepatitis C virus |
| HIV | human immunodeficiency virus |
| hr. | hour |
| HR+ | hormone receptor-positive |
| HREC | Human Research Ethics Committee |
| IB | Investigator's Brochure |
| ICF | informed consent form |
| ICH | International Council for Harmonisation of Technical |
| | Requirements for Pharmaceuticals for Human Use |
| INR | International Normalized Ratio |
| IP | investigational product |
| ITT | intent-to-treat |
| NYHA | New York Heart Association |
| MedDRA | Medical Dictionary for Regulatory Activities |
| MENQOL | Menopause-specific Quality of Life (questionnaire) |
| OTC | over-the-counter |
| Pap | Papanicolaou (test) |
| PD | pharmacodynamics |
| PE | pulmonary embolism |
| PK | pharmacokinetics |
| PT | prothrombin time |
| RBC | red blood cell |
| SADR | serious adverse drug reaction |
| SAE | serious adverse event |
| SAP | statistical analysis plan |
| SD | standard deviation |
| SERM | selective estrogen receptor modulator |
| THC | tetrahydrocannabinol |
| TIA | transient ischemic attack |
| t_{1/2} | plasma terminal half-life |
| Tₘₐₓ | time of occurrence of maximum observed |
| | plasma drug concentration |
| TSH | thyroid-stimulating hormone |
| UTI | urinary tract infection |
| VVA | vulvar and vaginal atrophy |
| WHO-DD | World Health Organization Drug Dictionary |

### Examples

The following examples are intended to illustrate certain embodiments of the present disclosure, but do not exemplify the full scope of the disclosure.

### Example 1: Phase 1/2 Study of Intravaginal Tamoxifen: Randomized, Double-Blind, Placebo-Controlled Study of Safety, Pharmacokinetics and Pharmacodynamics in Postmenopausal Participants with Moderate to Severe Vulvar and Vaginal Atrophy

### Study Design

This is a study to evaluate the safety, pharmacokinetics (PK) and pharmacodynamics (PD) of intravaginal tamoxifen in postmenopausal participants with moderate to severe vulvar and vaginal atrophy (VVA). The study objectives are:

### Primary:

To evaluate the safety and tolerability of tamoxifen by intravaginal administration
To determine the plasma pharmacokinetics (PK) of intravaginal tamoxifen after intravaginal application

### Secondary:

To evaluate preliminary efficacy and pharmacodynamics (PD) of intravaginal tamoxifen in terms of most bothersome symptom(s), dyspareunia, and changes in vaginal cytology and pH

### Exploratory:

To evaluate the impact of intravaginal tamoxifen on quality of life using a menopause-specific instrument
To assess the usability and acceptability of intravaginal tamoxifen.

TABLE 1
Objectives and Endpoints

| | |
|---|---|
| Objectives | Endpoints |

Primary

| | |
|---|---|
| To evaluate the safety and tolerability of a | Adverse events |
| tamoxifen vaginal insert by intravaginal | Clinical laboratory findings |
| administration | Vital signs results |
| | 12-lead ECG results |
| | Physical examination findings |
| | Change in local site irritation scores |
| | from baseline to Day 57 |
| | Endometrial thickness (measured by |
| | transvaginal ultrasound) in women who have |
| | not undergone hysterectomy prior to the |
| | study |
| To determine the plasma PK of tamoxifen | Standard PK parameters from |
| after intravaginal application | non-compartmental analysis on Day 1, |
| | with steady-state parameters on Day 56 |
| | Dose-proportionality of tamoxifen |

Secondary

| | |
|---|---|
| To evaluate preliminary efficacy and | Change of vaginal pH from baseline to Day |
| pharmacodynamics of tamoxifen vaginal | Change in vaginal cytology (maturation |
| insert in terms of most bothersome | index; percentage of basal and superficial |
| symptom, dyspareunia, and changes in | cells) from baseline to Day 57 |
| vaginal cytology and pH | Change in most bothersome symptom |
| | from baseline to Day 56 |
| | Change in severity of dyspareunia |
| | from baseline to Day 56 |
| | Correlation between exposure and |
| | local changes |

Exploratory

| | |
|---|---|
| To evaluate the impact of tamoxifen | Change in the MENQOL questionnaire |
| vaginal insert on quality of life using a | from baseline to Day 57 |
| menopause-specific instrument | Responses to the Usability and |
| To assess the usability and acceptability of | questionnaire over time |
| tamoxifen vaginal insert | |

This study is a Phase 1/2, randomized, multi-center, double-blind, parallel-arm, placebo-controlled, dose-ranging study to evaluate the safety, tolerability, PK, and PD of a tamoxifen vaginal insert. The investigational product (IP) will be evaluated at 4 dose levels (1 mg, 5 mg, 10 mg, and 20 mg) and compared to a placebo vaginal insert. The IP will be administered intravaginally for 56 days according to the following treatment schedule: once daily for 2 weeks then twice a week for 6 weeks.

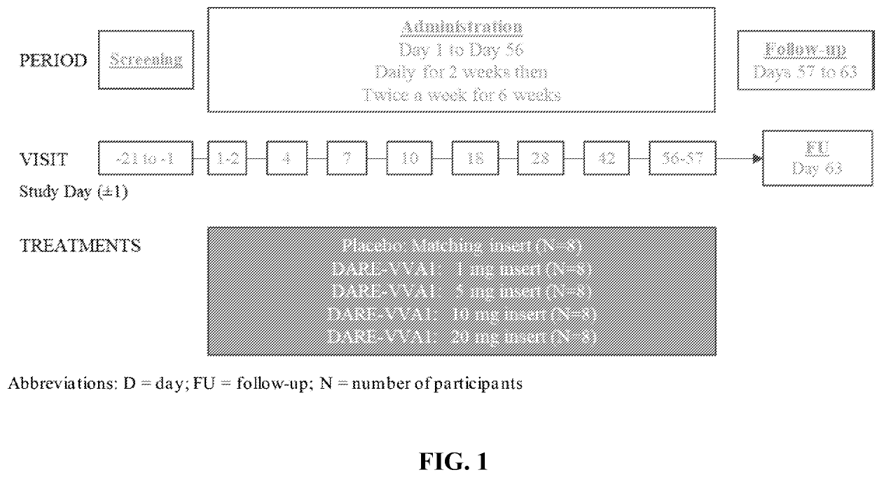

Approximately 40 postmenopausal women with Vulvar and Vaginal Atrophy (VVA) will be enrolled, 20 who have undergone hysterectomy and 20 who have not. After signing informed consent, eligible participants will be randomly allocated as follows: the 20 hysterectomized women will be allocated to one of the 5 treatment groups, and the 20 non-hysterectomized women will be randomly allocated to one of the same 5 treatment groups, such that each treatment group ends up with 4 hysterectomized women and 4 non-hysterectomized women, for a total of 8 women. In each treatment group, participants will have serial blood sampling for PK analysis and undergo safety evaluations and preliminary assessments of effectiveness. Following the completion of treatment, participants will attend a follow-up visit on Day 63 (FIG. 1).

Preliminary efficacy will be assessed by vaginal cytology (for maturation index) and by vaginal pH, measured at baseline, every 2 weeks during treatment, following completion of treatment (Day 57), and at the follow-up visit conducted on Day 63. In addition, most bothersome symptom and dyspareunia will be evaluated during the treatment period and at follow-up. An exploratory assessment of quality of life will be conducted using the Menopause-specific Quality of Life (MENQOL) questionnaire, and participants will report on the usability and acceptability of a tamoxifen vaginal insert using a questionnaire.

In each treatment group, participants will have serial blood sampling for PK analysis, with venous samples drawn from an indwelling line. Dense sampling will be performed following the Day 1 and Day 56 administrations of a tamoxifen vaginal insert; trough samples will be collected prior to dosing on Days 4, 7, 10, 18, 28, and 42, and a final sample will be collected at follow-up on Day 63. Samples will be analyzed using validated methods to determine plasma concentrations of tamoxifen and 3 metabolites: 4-hydroxytamoxifen, N-desmethyl-tamoxifen, and N-desmethyl-4-hydroxytamoxifen (endoxifen).

Safety will be assessed by evaluating AEs, vital sign measurements, clinical laboratory test results, 12-lead ECGs, and physical examination findings. In addition, vaginal speculum examinations will be performed, including an assessment of local irritation (in terms of erythema and edema). Women without a history of hysterectomy will undergo transvaginal ultrasounds at screening and at the end of treatment (Day 57).

Following the completion of treatment, participants will attend a follow-up visit on Day 63. All AEs observed by the study personnel or reported by the participant during the study (from the time of the signing of the informed consent through the follow-up visit) will be documented.

### Participant Treatment Groups

This study will enroll sufficient postmenopausal female participants with moderate to severe VVA to randomly allocate 40 participants, 20 who have undergone hysterectomy and 20 who have not, to receive treatment with intravaginal tamoxifen (4 dose levels) or placebo. A sufficient number of participants will be screened to randomize 40 participants (8 participants per group) to the following planned treatment groups:

TABLE 2

| | | Number of |
|---|---|---|
| Dose Level | Treatment | Participants |
| Placebo | Placebo insert | N = 8 |
| 1 mg | intravaginal tamoxifen 1 mg insert | N = 8 |
| 5 mg | intravaginal tamoxifen 5 mg insert | N = 8 |
| 10 mg | intravaginal tamoxifen 10 mg insert | N = 8 |
| 20 mg | intravaginal tamoxifen 20 mg insert | N = 8 |

This study will enroll healthy postmenopausal females aged 40-75 years with moderate to severe VVA. Participants must meet all inclusion criteria to be eligible for enrollment into the study. Participants will not be eligible for entry into this study if they meet any of the exclusion criteria and will be discontinued at the discretion of the investigator in consultation with the medical monitor if they develop any of the exclusion criteria during the study.

With the exception of all prohibited therapies described below, the use of concomitant medications is allowed, but should be limited to those medications considered necessary. The use of vaginal lubricants is permitted; however, they should not be used within 6 hours of administration of the IP.

### Inclusion Criteria:

1. Women aged 40-75 (inclusive).
2. Postmenopausal women with a body mass index between 18 and 38 kg/m², inclusive.
3. Postmenopausal, defined as:
   a. For non-hysterectomized women, 12 months of spontaneous amenorrhea or 6 months of spontaneous amenorrhea with serum follicle-stimulating hormone (FSH) levels>40 mIU/mL or 6 weeks postsurgical bilateral oophorectomy.
   b. For hysterectomized women, serum follicle-stimulating hormone (FSH) levels>40 mIU/mL or 6 weeks postsurgical bilateral oophorectomy.
4. Have moderate to severe VVA as determined by self-assessment of at least one the following symptoms: i) vaginal dryness, ii) vaginal and/or vulvar irritation/itching, iii) dysuria, iv) vaginal pain with sexual activity (dyspareunia) (all self-assessed as none, mild, moderate, or severe), or vaginal bleeding associated with sexual activity (self-assessed as presence versus absence). To be eligible, at least 1 of the first 4 symptoms must be reported as moderate or severe, or vaginal bleeding associated with sexual activity must be present.
5. Women who currently have vaginal intercourse or other sexual activity (masturbation, etc.) at least once a month (with or without a partner), or who had intercourse or other sexual activity at least once a month in the past, but later decreased sexual activity due to excessive pain or vaginal dryness. Participants must be willing to engage in vaginal intercourse or other sexual activity (masturbation, etc.) at least 1 time between Days 49-56 of the clinical study.
6. Participants, upon pelvic examination with speculum examination, must have a normal-appearing vulva other than atrophic changes and normal-appearing vagina (without erosions, ulcerations, scarring, or evidence of dermatoses) other than atrophic changes (loss of rugae, mucosal pallor, mucosal dryness, mucosal petechiae). Women without a cervix must have a normal-appearing vaginal cuff.
7. Women who have not undergone hysterectomy must have:
   a. No prior history of endometrial ablation.
   b. Endometrial thickness≤4 mm on transvaginal ultrasound.
8. Women with a cervix must:
   a. Have a normal-appearing cervix other than atrophic changes (i.e., cervical stenosis and/or flushness with the vaginal wall).
   b. Be current on all recommended screening and management requirements for cervical cancer.
9. Vaginal cellular cytology with ≤5% superficial cells.
10. Vaginal pH>5 at Screening Visit.
11. Normal mammogram report within 2 years of screening.
12. Normal manual breast examination by investigator at baseline.
13. Baseline hematology, clinical chemistry, urinalysis, coagulation, and viral serologies for human immunodeficiency virus (HIV), hepatitis C virus (HCV), and hepatitis B surface antigen (HBsAg) all within normal limits OR accepted by the investigator and medical monitor as not clinically significant.
14. Normal 12-lead electrocardiogram (ECG).
15. Able to read, understand, and provide written informed consent and applicable data protection authorization after the nature of the study has been fully explained, and must be willing to comply with all study requirements.
16. Willing and able to correctly and independently complete all study procedures.

Eligible participants who have not undergone hysterectomy will have no history of endometrial ablation or endometrial hyperplasia, with ultrasound at screening showing endometrial thickness≤4 mm. Participants with a cervix will also have an up-to-date cervical cancer screening record. All participants must have a normal mammogram within 2 years of screening; participants who have not had a mammogram in the last 2 years, or have a cervix but have not had a cervical screening test (CST) in the last 5 years, will be required to undergo these assessments during screening.

### Exclusion Criteria:

1. A history of or physical examination finding for any significant cardiovascular, renal, pulmonary, neurological and hepatic diseases preventing compliance with this study.
2. A medical history of or use of anticoagulant drugs to treat or prevent coagulopathies, thrombophilia or thromboembolic disease (deep vein thrombosis, pulmonary or systemic embolism, stroke, or transient ischemic attack).
3. Uncontrolled hypertension (either systolic>180 mmHg or diastolic>105 mmHg), treatment with Class 1 antiarrhythmics or digitalis, history of congestive heart failure (New York Heart Association [NYHA]>Class I), or myocardial infarction within 12 months.
4. Women with a cervix cannot have had an abnormal cervical screening test within 2 years of screening. Participants can have atypical squamous cells of undetermined significance if human papilloma virus-negative.
5. Women who have not undergone hysterectomy cannot have a history of or current endometrial pathology: hyperplasia, carcinoma and/or polyp (prior history of a benign endometrial polyp with no current evidence of polyp is acceptable).
6. A medical history of breast cancer within 5 years of screening. Participants with a history of breast cancer more than 5 years prior to screening are considered eligible if their disease was node-negative, nonmetastatic, and if all treatment with aromatase inhibitors (AIs) or SERMs was completed at least 6 months prior to screening.
7. A medical history of malignant melanoma.
8. Any cancer (except nonmelanomatous skin cancer) diagnosed less than 5 years prior to the Screening Visit.
9. A medical history of undiagnosed vaginal bleeding.
10. A known or suspected estrogen-dependent neoplasia.
11. Previous radiation treatment to the pelvis.
12. Women who have previously reported an unsatisfactory outcome from a vaginal hormone therapy for VVA.
13. Known hypersensitivity to any ingredients in DARE-VVA1.
14. Use of vaginal hormonal products (rings, creams, gels, tablets, capsules) within 4 weeks prior to Day 1.
15. Use of transdermal estrogen products within 4 weeks prior to Day 1.
16. Use of oral estrogen therapy within 8 weeks prior to Day 1.
17. Use of estrogen-alone injectable drug therapy within 12 weeks prior to Day 1.
18. Administration of estrogen pellet therapy within 6 months prior to Day 1.
19. Use of thyroid hormone replacement therapy unless the participant is on a stable dose for >6 months, and participant is euthyroid based on a normal, sensitive immunoassay for thyroid-stimulating hormone (TSH).
20. Use of SERMs or AIs within 6 months prior to screening.
21. Use of anabolic or other steroids (including hormonal creams such as testosterone) within 4 weeks prior to Day 1.
22. Use of corticosteroids, >5 mg/day prednisone or equivalent, for more than 4 weeks within 4 weeks prior to Day 1.
23. Participants with any self-reported active sexually transmitted disease and/or evidence of infection (including bacterial vaginosis) on vaginal examination by the investigator.
24. Participants with a urinary tract infection during screening as assessed by urine dipstick test with abnormal test findings (any positive result for leukocytes AND any positive result for nitrites).
25. Women who have not undergone hysterectomy cannot have clinically significant uterine fibroids.
26. Evidence of current alcohol or drug abuse in the past 60 days, including a positive result from the urine drugs of abuse or alcohol screen, or history of drug or alcohol dependence in the last 2 years, as assessed by the investigator. Alcohol abuse is defined as greater than 14 standard units/week for females, and drug abuse is defined as known psychiatric or substance abuse disorder that would interfere with participation with the requirements of this study, including current use of any illicit drugs. Use of medical cannabis is not exclusionary.
27. Participation in any other investigational drug or device trial in which administration of an investigational study drug/device occurred within 30 days or placement of a non-drug eluting medical device within 15 days prior to the Screening Visit (Visit 1).
28. In the opinion of the investigator, participant has any disorder or finding that might interfere with the conduct of the study.

Participants will be excluded if they have any of the following: a history of coagulopathies, thrombophilia, thromboembolic disease, or significant transient ischemic attack; significant cardiac history; recent history of breast cancer (or suspicion of breast cancer), abnormal cervical screening result within 2 years; endometrial pathology; known or suspected estrogen-dependent neoplasia. Safety monitoring will include 12-lead electrocardiograms (ECGs) to evaluate possible cardiac problems; laboratory assessments will include measurement of liver functions tests and clotting parameters, to monitor hepatic safety and potential embolism/thrombotic events, respectively; and regular vaginal inspection. Due to the reported risks of endometrial adenocarcinoma and uterine sarcoma, transvaginal ultrasounds will be performed at screening on women who have not undergone hysterectomy in order to measure endometrial thickness.

Participants will also be excluded if they are receiving medications that could confound assessments (e.g., vaginal hormonal products, estrogen therapy, or use of SERMs or AIs within 6 months prior to screening) or have previously experienced an unsatisfactory outcome from a vaginal hormone therapy for VVA.

The following therapies are prohibited both during the study and in the period before the study as shown:
use of anticoagulant drugs to treat or prevent coagulopathies, thrombophilia, thromboembolic disease, or transient ischemic attack
radiation treatment to the pelvis
vaginal hormonal products (rings, creams, gels, tablets, capsules) (within 4 weeks prior to Day 1)
transdermal estrogen products (within 4 weeks prior to Day 1)
oral estrogen therapy (within 8 weeks prior to Day 1)
estrogen-alone injectable drug therapy (within 12 weeks prior to Day 1)
estrogen pellet therapy (within 6 months prior to Day 1)
thyroid hormone replacement therapy is prohibited unless the participant has been on a stable dose for >6 months, and is euthyroid based on a normal, sensitive immunoassay for TSH
SERMS or AIs (within 6 months prior to Day 1)
anabolic or other steroids (including hormonal creams such as testosterone) (within 4 weeks prior to Day 1)
corticosteroids, >5 mg/day prednisone or equivalent, for more than 4 weeks within 4 weeks prior to Day 1
investigational drug (within 30 days prior to screening) or non-drug eluting medical device (within 15 days prior to screening)

The following therapies are prohibited during the study:
intravaginal products (e.g., Femring® [estradiol acetate vaginal ring], ESTRING® [estradiol vaginal ring]) or medication, OTC or prescription

NOTE: the use of vaginal lubricants is permitted, but not within 6 hours of administration of the IP
consumption of St John's Wort (hypericum perforatum) and grapefruit, Seville (bitter) orange, and pomelo fruit/juice/preserves
the following CYP3A4/5 inducers and CYP2D6 inhibitors are prohibited: CYP3A4/5 inducers: Apalutamide (strong), aprepitant, armodafinil, Bosentan (moderate), Carbamazepine (strong), corticosteroids, Dabrafenib, Efavirenz (moderate), encorafenib, enzalutamide (strong), etravirine (moderate), Loriatinib (moderate), lumacaftor (strong) Modafinil (moderate) Nevirapine Phenobarbital, phenytoin (strong), Rifabutin, rifampicin (strong), ritonavir, rufinamide, Tipranavir, Vemurafenib.
   CYP2D6 inhibitors: Abiraterone, amiodarone, Bupropion (strong), Celecoxib, cinacalcet (moderate), cobicistat, Duloxetine (moderate), Fluoxetine (strong), Methadone, mirabegron (moderate), Paroxetine (strong), Terbinafine (strong)

### Investigational Product (IP)

The investigational product (IP) of the present study is a tamoxifen citrate soft gelatin capsule-based vaginal insert comprising 1, 5, 10, or 20 mg tamoxifen administered vaginally with self-placement by the subject. The control product of the present study is a placebo vaginal insert administered vaginally with self-placement by the subject. It is intended for once-daily administration for 2 weeks followed by twice-weekly dosing, with self-placement by the patient. All capsules are 19.0 long×11.4 mm wide.

### Formulation

Formulation development activities included the design of new formulations (including solubility screen and excipient compatibility testing), and assessments of physical stability, chemical stability, and in-vitro dissolution in simulated vaginal fluid using fiber optic apparatus. Based on the physical and chemical stability and on the dissolution profile of prototypes in simulated vaginal fluid, a prototype was selected for non-GMP batch manufacturing. The selected formulation was composed of tamoxifen citrate, Hard Fat (Wecobee FS), Mineral Oil, USP, and White Petrolatum, USP.

Prototype lots were placed on stability at 25° C./60% RH and 40° C./75% RH. The prototype tamoxifen 1.0 mg, 20 mg, and placebo capsules are size 20 Oval off-white soft gelatin capsules containing 1000 mg of fill material (fill material range 970-1030 mg). The shell formula contains glycerin (plasticizer), Type 150 bloom lime processed gelatin (bovine bone), and purified water. The gel contained a minimal amount of titanium dioxide as the opacifier.

TABLE 3
Composition of Investigational Product Soft Gelatin Capsule Inserts

| | Tamoxifen | | Tamoxifen | | Tamoxifen | | Tamoxifen | | Tamoxifen | |
|---|---|---|---|---|---|---|---|---|---|---|
| | Placebo Inserts | | 20 mg Inserts | | 10 mg Inserts | | 5 mg Inserts | | 1 mg Inserts | |
| | (OET-10275757) | | (OET-10275759) | | (OET-10275812) | | (OET-10275781) | | (OET-10275758) | |
| Name of Material | mg/Insert | % w/w | mg/Insert | % w/w | mg/Insert | % w/w | mg/Insert | % w/w | mg/Insert | % w/w |
| Tamoxifen Citrate | — | — | 30.342 | 3.034 | 15.171 | 1.517 | 7.585 | 0.759 | 1.517 | 0.152 |
| Wecobee FS | 271.932 | 27.193 | 263.648 | 26,365 | 267.800 | 26.788 | 269.870 | 26.987 | 271.522 | 23.152 |
| White petroleum, USP | 271.932 | 27.193 | 263.648 | 26.365 | 267.800 | 26.780 | 269.870 | 26.987 | 271.522 | 27.152 |
| Mineral off, USP | 356.136 | 45.614 | 442.362 | 44.236 | 449.229 | 44.923 | 452.675 | 45.268 | 435.439 | 45.544 |
| Total weight | 1000.000 | 100.000 | 1000.000 | 100.000 | 1000.000 | 100.000 | 1000.000 | 100.000 | 1000.000 | 100.000 |

Abbreviations. USP = United States Pharmacopoeia

### Treatment Regimen

The tamoxifen vaginal inserts and placebo inserts will be inserted by the participant in the morning. Participants should make every effort to ensure that inserts are inserted at approximately the same time for each administration except on visit days when IP should be inserted after trough PK sample collection.

Participants will be instructed on how to correctly self-administer the IP on Day 1, at the time of the first administration, and a warning should be given that the insert should not be pushed up too close to the cervix. The IP is to be self-administered by the patient, intravaginally, on a daily or twice-weekly basis. Patients can administer the insert in one of several positions: lying down, squatting, or bending over with one leg raised. Patients will be advised to wash hands thoroughly before and after insertion.

On Days 1 and 56, blood samples for PK analysis will be collected immediately before administration of the IP, and at scheduled intervals after administration in accordance with the sampling schedule defined in the schedule of events (Table 4). In addition, blood samples will be collected within 30 minutes before administration of the tamoxifen vaginal insert on Days 4, 7, 10, 18, 28, and 42. A final sample will be collected at follow-up on Day 63.

Throughout the 56-day administration period, participants will be asked to record their administration of the tamoxifen vaginal insert on a paper diary, including the time of placement. Deviations from the planned doses (missed dose or timing) will be recorded on the participant's eCRF. Study personnel will review diaries at each visit, and diaries will be collected as source documents. Information from participant diaries will be transcribed on the appropriate eCRF pages for documentation of participant compliance with the IP.

Participants will be required to engage in vaginal intercourse or other sexual activity (masturbation, etc.) at least 1 time between Days 49-56 of the clinical study.

Tamoxifen vaginal inserts and placebo inserts will be self-administered by participants for 56 days (Days 1 to 56). The IP will be inserted intravaginally in the morning, at approximately the same time each day. Treatment will be administered once a day for the first 2 weeks, followed by twice weekly treatment for 6 weeks. The first twice-weekly dose will be administered on Day 18 during a study visit.

The sequence and maximum duration of the study periods will be as follows:
1. Screening: 21 days
2. Treatment: 56 days
3. Follow-up: 7 days

The maximum treatment duration for each participant is 56 days. The maximum study duration for each participant is approximately 12 weeks.

### Screening:

The participant will be screened within 21 days before enrollment in the study. Assessments to be conducted during screening are shown in the schedule of events (Table 4). No study-specific assessments will be conducted until the participant has provided written informed consent. Participants who have not had mammograms and CSTs in the timeframe indicated by the inclusion criteria will undergo these assessments during screening.

### Double-Blind Treatment Period:

The treatment period is defined as the period from Day 1 to Day 57, covering the 24-hour period after the final administration of the IP on Day 56. Participants will self-administer the tamoxifen vaginal inserts and placebo inserts as instructed between Days 1 and 56, in accordance with the treatment group to which they are allocated. Study assessments will be conducted in accordance with the schedule of events (Table 4).

### Day 1 to 2

The Day 1 to Day 2 visit will be an overnight stay at the study site. Participants will attend the study site on the morning of Day 1. Participants will be instructed on how to correctly self-administer the IP at the time of the first administration, and a warning should be given that the insert should not be pushed up too close to the cervix.

Samples for analysis of tamoxifen PK will be collected before administration of the IP and at the scheduled times indicated in the schedule of events (Table 4). Where multiple assessments are scheduled for the same time point, assessments should be performed within 15 minutes of the scheduled time with priority given to any PK blood sample procedures, if applicable.

Participants will be provided with a diary in which to record their administration of the tamoxifen vaginal inserts and placebo inserts including the time of placement, and will be trained on its completion. Participants will bring their diaries to all site visits for review. Diaries will be completed at site on days where the tamoxifen vaginal insert and placebo insert is administered at site.

On Day 2, after participants have provided the 24-hour PK sample, a vaginal speculum examination will be conducted before the participant self-administers the second IP insert. Participants will be discharged from the study site on Day 2, following collection of the 24-hour PK sample and completion of all 24-hour assessments and insertion of the second IP insert. At discharge, participants will be provided with sufficient IP to last for the 56-day treatment period.

### Days 4, 7, 10, 18, 28, and 42

Participants will return to the study site on Days 4, 7, 10, 18, 28, and 42, to complete the procedures shown in the schedule of events (Table 4). On Days 4, 7, 10, 18, 28, and 42, participants will not self-administer the IP at home; blood samples for trough PK assessments will be collected at site within 30 minutes before the IP is administered. Participants will bring their diaries to each of these visits.

### Day 56 to 57

The Day 56 to Day 57 visit will be an overnight stay at the study site. Participants will attend the study site in the morning of Day 56. Participants will not self-administer the IP at home on Day 56; the IP will be administered at the site as instructed. Any unused IP should be returned to the study site. Samples for analysis of tamoxifen PK will be collected before administration of the IP and at the scheduled times indicated in the schedule of events (Table 4). Where multiple assessments are scheduled for the same time point, assessments will be performed within 15 minutes of the scheduled time with priority given to any PK blood sample procedures, if applicable. Participants will be discharged from the study site after completion of all 24-hour assessments noted on Day 57.

### Day 63 Follow-Up Evaluation

At 7 days after the last administration of the IP, the participants will return to the study site for a follow-up visit. Safety and efficacy assessments will be conducted in accordance with the schedule of events (Table 4), and a final PK sample will be collected.

TABLE 4
Schedule of Events

| | Screeni | Administration Period | | | | | | | | | | | | | | | | ET | FU |
|---|---|---|---|---|---|---|---|---|---|---|---|---|---|---|---|---|---|---|---|
| Day(s) | −21 to −1 | 1-2 [symbol] | | | | | [symbol] b | [symbol] a, b | 10 [symbol] | 18 [symbol] | 28 [symbol] | 42 [symbol] | 56-57 [symbol] | | | | | [symbol] | 63 |
| Window | — | — | | | | | = 1 | = 1 | = 1 | = 1 | = 1 | = 1 | = 1 | | | | | [symbol] | = 1 |
| Time from Admin. | | Pre- | 0 | 4 | 10 | 24^{c} | | | | | | | Pre- | 0 | 4 | 10 | 24^{c} | | |
| Informed consent | X | | | | | | | | | | | | | | | | | | |
| Inclusion/Exclusion | X | | | | | | | | | | | | | | | | | | |
| Medical history | X | | | | | | | | | | | | | | | | | | |
| Concomitant | X | X | | | | | X | X | X | X | X | X | X | | | | X | X | X |
| Physical exam^{d} | X | X | X | X | X | X | X | X | X | X | X | X | X | X | X | X | X | X | X |
| Vaginal speculum | X | X | | | | X | | X | | X | X | X | | | | | X | X | X |
| Mammogram [symbol] | X | | | | | | | | | | | | | | | | | | |
| Cervical screening | X | | | | | | | | | | | | | | | | | | |
| Height and weight | X | | | | | | | | | | | | | | | | | | |
| Vital signs [symbol] | X | X | | X | X | X | | X | | X | X | X | X | | X | X | X | X | X |
| 12-Lead ECG [symbol] | X | X | | | | | | X | | X | X | | X | | X | | X | X | X |
| Serum biochemistry | X | X | | | | | | X | | X | X | | | | | | X | X | X |
| Hematology | X | X | | | | | | X | | X | X | | | | | | X | X | X |
| Urinalysis | X | X | | | | | | X | | X | X | | | | | | X | X | X |
| Coagulation panel | X | X | | | | | | X | | X | X | | | | | | X | | X |
| TSH | X | | | | | | | | | | | | | | | | | | |
| FSH | X | | | | | | | | | | | | | | | | | | |
| Drugs of abuse [symbol] | X | | | | | | | | | | | | | | | | | | |
| Viral seology [symbol] | X | | | | | | | | | | | | | | | | | | |
| Vaginal pH [symbol] | X | X | | | | | | | | X | X | X | | | | | X | X | X |
| Vaginal cytology | X | X | | | | | | | | X | X | X | | | | | X | X | X |
| Transvaginal | xn | | | | | | | | | | | | | | | | X | X | |
| Assessment of VVA | X | | | | | | | | | X | X | X | X | | | | | X | X |
| symptoms [symbol] | | | | | | | | | | | | | | | | | | | |
| Diary administration | X | X | | | | | X | X | X | X | X | X | X | | | | | X | |
| and review [symbol] | | | | | | | | | | | | | | | | | | | |
| Study drug | | | X | | | X | X | X | X | X | X | X | | X | | | | | |
| MENQOL | | X | | | | | | | | | | | | | | | X | X | |
| Usability and | | | | | | X | | | | | | X | | | | | X | X | |
| Acceptability | | | | | | | | | | | | | | | | | X | | |
| PK blood samples [symbol] | | X | 0.5, 1, 2, 4, 5, | | | X | X | X | X | X | X | X | X | 0.5, 1, 2, 4, 5, | | | X | | X |
| | | | 8, 12 (hr.) | | | | | | | | | | | 8, 12 (hr.) | | | | | |
| AE [symbol] | | X | X | X | X | X | X | X | X | X | X | X | X | X | X | X | X | X | X |

Abbreviation: AE = adverse event; ECG = electrocardiogram; ET = early termination; FSH = follicle-stimulating hormone; FU = follow-up; HBsAg = hepatitis B surface antigen; HCV = hepatitis C virus; HIV = human immunodeficiency virus; hr = hour; IP = investigational product; MENQL = Menopause-specific Quality of Life; PK = pharmakokinetics; TSH-thyroid-stimulating hormone; VVA = vulvar vaginal atrophy [symbol]
indicates data missing or illegible when filed

a. Multiple non-PK assessments scheduled for the same time point should be performed on the same day as dosing. PK blood sample procedures should follow the defined window, if applicable.
b. Samples for PK trough levels should be obtained prior to tamoxifen vaginal insert insertion on Days 4, 7, 10, 18, 28, and 42. Administration of tamoxifen vaginal insert will take place at site on the day of these visits, after collecting the trough PK sample.
c. Participants will be discharged from the site after all 24-hour assessments have been completed. Day 1-2 only: Administration of tamoxifen vaginal insert should be the final activity before discharge, following completion of all 24-hour assessments.
d. A complete physical examination (excluding rectal examinations) will be performed at screening and will include a manual breast examination. Subsequent physical examinations will be abbreviated and will be directed based on signs and symptoms exhibited by the participant.
e. Vaginal speculum examinations will include assessment of local irritation (scoring of erythema and edema), and a full pelvic examination.
f. Mammograms should be conducted ONLY in those participants who have not had one within the last 2 years.
g. Cervical screen testing should be conducted ONLY in those participants who have a cervix and have not had cervical screening within the last 5 years.
h. Vital signs assessments will include pulse rate, respiratory rate, and supine blood pressure. Assessments will be performed after the participant has been in a supine position for at least 5 minutes. Temperature will also be measured.
i. 12-lead ECGs will be performed after the participant has been supine for at least 5 minutes.
j. FSH will be assessed in all hysterectomized women who have not undergone bilateral oophorectomy, and in non-hysterectomized women who have not undergone bilateral oophorectomy and have experienced spontaneous amenorrhea for ≥6 months but <12 months, to confirm postmenopausal status.
k. Drugs of abuse will be measured at screening by local laboratories via urinalysis and alcohol screens versus a commercially available urine dipstick.
l. Viral serology: a blood sample will be collected for the determination of HIV, HCV, and HBsAg.
m. Vaginal pH will be evaluated using commercially available pH strips.
n. Transvaginal ultrasound will be performed on women who have not undergone a hysterectomy. During screening, the pelvic examination, with assessment of local irritation and cervical screening (if needed), should be performed prior to the transvaginal ultrasound.
o. Participants will be required to engage in vaginal intercourse or other sexual activity (masturbation, etc.) at least 1 time between Days 49-56, and complete an assessment of VVA symptoms in their diary.
p. Diaries will be provided to participants at screening, and participants will be trained on their completion. Participants will record their administration of tamoxifen vaginal insert in the diary, including the time of placement. Study staff should instruct participants to bring their diaries to all site visits for review.
q. On Days 4, 7, 10, 18, 28, and 42, participants should not self-administer the IP at home; blood samples for trough PK assessments should be collected at site before the IP is administered.
r. Permissible windows for PK sampling are as follows: 0.5 hours and 1 hours=±10 minutes; 2, 4, 5, 8, 12, and 24 hours==15 minutes. Trough samples collected on Days 4, 7, 10, 18, 28, and 42 should be collected within 30 minutes before IP administration.
s. Adverse event information will be collected at the specified time points, as well as at any time when a clinical research unit staff member becomes aware of an AE during the administration period.

### Evaluation of Objectives

### Pharmacokinetics:

Following baseline evaluations including the time 0 venous plasma sample, the Day 1 tamoxifen vaginal insert dose or matching placebo will be administered. Serial peripheral venous samples will be drawn from an indwelling line at 0.5, 1, 2, 4, 5, 8, 12, and 24 hours. The same sampling schedule will be used for PK analysis with administration of the Day 56 vaginal insert. Trough samples will be collected before administration of inserts on Days 4, 7, 10, 18, 28, and 42, and a final sample will be collected at follow-up on Day 63.

Samples will be analyzed using validated methods to determine plasma concentrations of tamoxifen and 3 metabolites: 4-hydroxytamoxifen, N-desmethyltamoxifen, and N-desmethyl-4-hydroxytamoxifen (endoxifen).

The PK characterization of tamoxifen concentrations for each dose to be profiled will use noncompartmental analysis (NCA). Three tamoxifen metabolites (4-hydroxytamoxifen, N-desmethyltamoxifen, and N-desmethyl-4-hydroxytamoxifen [endoxifen]) will also undergo PK characterization. Standard PK parameters assessed will include measures of the extent of absorption using estimates of the area under the plasma concentration-time curve (AUC), the maximum observed drug concentration (Cmax), and the time to reach maximum drug concentration (Tmax). Additional details of the parameters and their calculation and evaluation will be included in the statistical analysis plan (SAP).

The PK parameter estimates will be completed using WinNonlin software. Actual sampling time will be used for all parameter estimation. PK parameters that will be computed for each participant for samples obtained over the planned sampling intervals of 24 hours after administration of the IP on Days 1 and 56, with trough samples collected before dosing on Days 4, 7, 10, 18, 28, and 42, and a final sample on Day 63.

### Pharmacokinetic Parameters

Cₘₐₓ: maximum observed drug concentration
Tₘₐₓ: time to reach maximum (peak) drug concentration
AUC₀₋ₜ: area under plasma concentration-time curve from zero to time t
AUC_{0-inf}: area under plasma concentration-time curve from time zero to infinity
T_{1/2}: elimination half-life
λ_{z}: elimination rate constant
V_{d}: apparent volume of distribution
CL/F: apparent clearance

Dense PK sampling will be performed on Day 1 and Day 56, with samples collected immediately before insertion of the IP and at the following time points after insertion:
0.5 hours and 1 hour (±10 minutes)
2, 4, 5, 8, 12, and 24 hours (±15 minutes)
Trough samples (Days 4, 7, 10, 18, 28, and 42) should be collected within 30 minutes before administration of IP inserts. A final sample will be collected at the Day 63 follow-up visit.

### Safety:

Observations of safety (self-reported or by examination) will be recorded on an ongoing basis. Any irritation, discharge or pain will be reported as an adverse event (AE) using standardized Medical Dictionary for Regulatory Activities (MedDRA) codes and graded for severity.

Visual examination by vaginal speculum and full pelvic examination for any vaginal inflammation will be conducted at screening, pretreatment (Day 1), during treatment, and 1 week after treatment. Clinical laboratory measurements including full hematology and biochemistry panels will be recorded at screening, at baseline, on Days 7, 18, 28, and 57, and 1 week after treatment.

Dipstick urinalysis of a void sample will be reported at screening, at baseline, on Days 7, 18, 28, and 57, and 1 week after treatment. If abnormal, the sample will be centrifuged for microscopic description of cellular elements. Vital signs, including pulse rate, respiratory rate, and supine blood pressure, will be recorded in a supine position at screening, during the administration period, and 1 week after treatment. Standard 12-lead ECGs will be recorded at screening, prior to dose on Days 1, 7, 18, 28, and 56, at 4 hours post-dose on Days 1 and 56, on Day 57, and 1 week after treatment.

A complete physical examination (excluding rectal examinations) will be performed at screening and will include a manual breast examination. Subsequent physical examinations will be abbreviated and will be directed based on signs and symptoms exhibited by the participant. Pelvic speculum and visual examinations will be conducted to identify vaginal abnormalities. Clinically significant abnormal findings from the speculum examination will be reported as an AE. Investigators will also conduct an assessment of local irritation; erythema and edema will be rated from 0 (none) to 3 (severe). Women who have not undergone hysterectomy will have a transvaginal ultrasound to determine endometrial thickness.

### Safety Assessments:

Safety assessments will include the evaluation of AEs, clinical laboratory assessments, vital signs, 12-lead ECGs, physical examinations, vaginal speculum examinations, and transvaginal ultrasounds.

### Clinical Laboratory Tests to be Performed:

Samples for the following laboratory tests will be collected at the time points specified in the schedule of events (Table 4).

Hematology: hemoglobin, hematocrit, red blood cell (RBC) count, RBC indices (mean corpuscular hemoglobin, mean corpuscular hemoglobin concentration, mean corpuscular volume, red blood cell distribution width), platelet count (or estimate), white blood cell count including differential

Serum Chemistry: albumin, total bilirubin, total protein, calcium, alkaline phosphatase, alanine aminotransferase, aspartate aminotransferase, blood urea nitrogen, creatinine, glucose, sodium, potassium, chloride, bicarbonate, lactate dehydrogenase, uric acid Coagulation Panel: PT, APTT, fibrinogen, INR

Urinalysis: Leukocytes, nitrite, pH, specific gravity, blood, glucose, protein, ketones, bilirubin, urobilinogen

Viral Serology: HIV, HCV, and HBsAg (screening only)

Urine Drug Screen: Urine drugs of abuse according to the local standard (including, at a minimum, cocaine, tetrahydrocannabinol, amphetamines and opiates) via urinalysis conducted by local laboratories, and alcohol screens via a commercially available urine dipstick. During the screening period, urine drugs of abuse and alcohol screens may not be repeated for eligibility unless the investigator believes that a positive result is possibly attributable to a concomitant medication; a repeat test will be allowed under this circumstance

Other: TSH (screening only), FSH (at screening only, to confirm postmenopausal status in nonhysterectomized women with spontaneous amenorrhea for ≥6 months but <12 months and no history of bilateral oophorectomy, and in hysterectomized women who have not undergone bilateral oophorectomy)

### Pharmacodynamics and Efficacy:

Estrogenic effect measurements will include vaginal cytology (maturation index; percentage of basal and superficial cells) and vaginal pH at screening and baseline, every 2 weeks during treatment, and 1 week after treatment.

Pharmacodynamics will be assessed by correlating the dose administered to dose exposure of tamoxifen in plasma, and then by relating the exposure to local vaginal changes in cytology and pH as well as to the reported symptomatology over 56 days.

### Vaginal pH

Vaginal swabs for determination of pH will be collected in accordance with the schedule of events (Table 4). The pH of vaginal secretions will be measured using pH paper.

### Vaginal Cytology

Samples for vaginal cytology will be collected at the time points specified in the schedule of events (Table 4) to determine the maturation index. Investigators will collect a swab of the lateral vaginal wall to be submitted for determination of the vaginal maturation index. The maturation index is determined by categorizing the ratio of the 3 types of vaginal epithelial cell (parabasal, intermediate, and superficial).

### Symptoms of Vulvar Vaginal Atrophy

At the Screening Visit, participants will rate the first 4 items in the list of VVA symptoms in Table 5 as either not present (none), mild, moderate, or severe, and will indicate whether vaginal bleeding associated with vaginal activity is present or absent. They will then select 1 of the 5 symptoms as their most bothersome symptom. All 5 symptoms will be evaluated again during later study visits (see Table 4), and the change in severity from baseline will be determined. Changes from baseline in each participant's most bothersome symptom, as well as changes in dyspareunia (whether dyspareunia was the participant's most bothersome symptom or not), will be evaluated as study endpoints. Symptoms to be evaluated are shown in Table 5. All participants will be required to engage in vaginal intercourse or other sexual activity (masturbation, etc.) at least 1 time between Days 49-56, and complete an assessment of VVA symptoms in their diary.

TABLE 5
Assessment of Most Bothersome Symptom

| Symptom | Assessment |
|---|---|
| Vaginal dryness | None, mild, moderate, or severe |
| Vaginal and/or vulvar | None, mild, moderate, or severe |
| irritation/itching | |
| Dysuria | None, mild, moderate, or severe |
| Vaginal pain associated | None, mild, moderate, or severe |
| with sexual activity | |
| Vaginal bleeding associated | Presence versus absence |
| with sexual activity | |

### Local Site Irritation

Pelvic speculum and visual examinations will be conducted to identify vaginal abnormalities. As part of this assessment, investigators will conduct an assessment of local irritation in terms of erythema and edema, as shown in Table 6.

TABLE 6

| Reaction Type | 0 = None | 1 = Mild | 2 = Moderate | 3 = Severe |
|---|---|---|---|---|
| Erythema | None | Faint or mild | Moderate redness | Intense redness |
| | | redness of any size | involving >25% of | involving >25% of |
| | | OR moderate | the area OR intense | the area |
| Edema | None | Mild visible | Moderate visible | Gross swelling or |
| | | swelling or barely | swelling OR easily | firm induration |
| | | palpable edema of | palpable edema | involving >25% |
| | | any size OR easily | involving >25% of | of the area |
| | | | [symbol] | |

[symbol]

indicates data missing or illegible when filed

### Menopause Quality of Life

The MENQOL™ questionnaire will be given to participants before the first dose and on Day 57, in accordance with the schedule of events (Table 4). Developed in 1996, the MENQOL is a self-administered, 29-item questionnaire with 4 domains: vasomotor, physical, psychosocial and sexual. Participants record whether they have experienced the listed problems in the past month, and rate the severity on a Likert scale from 0 (Not at all bothered) to 6 (Extremely bothered).

### Usability and Acceptability Questionnaire

A Usability and Acceptability questionnaire will be administered to all participants in accordance with the schedule of events (Table 4).

### Participant Diaries

Throughout the 56-day administration period, participants will be asked to record their administration of the tamoxifen vaginal inserts on a paper diary, including the time of placement. Deviations from the planned doses (missed dose or timing) will be recorded on the participant's eCRF. Participants will use their diaries to capture their assessment of VVA symptoms.

### Exploratory:

Menopause-specific Quality of Life (MENQOL) questionnaires will be completed prior to dosing on Day 1, and after treatment on Day 57. Details of the analysis of the responses to the Usability and Acceptability questionnaire will be outlined in the statistical analysis plan.

### Statistical Methods

### General:

For the purpose of all safety, efficacy, and other analyses where applicable, baseline is defined as the last measurement before the start of study drug administration. Continuous endpoints will be summarized with number (n), mean, standard deviation, median, minimum, and maximum. In addition, change from baseline values will be calculated at each time point and summarized descriptively. For categorical endpoints, descriptive summaries will include counts and percentages.

### Analysis Populations:

The ITT population, defined as all randomized participants, will be used to summarize participant disposition, demographics and baseline characteristic summaries, efficacy endpoints, and exploratory endpoints. Efficacy evaluations include changes in vaginal pH, changes in vaginal cytology (maturation index; percentage of basal and superficial cells), change in dyspareunia, and change in most bothersome symptom. These will be summarized using the ITT population. For the analysis the exploratory endpoints, results from the MENQOL questionnaire and the Usability and Acceptability questionnaire will be summarized over time from baseline in the ITT population.

An analysis of covariance (ANCOVA) model will be used to assess treatment differences in change from baseline for vaginal pH, vaginal cytology, and most bothersome symptom, using baseline score as a covariate. With no formal testing planned for this study, the P values will be informational.

The Safety population, defined all participants who are enrolled and receive any amount of planned IP, will summarize the recorded AEs, clinical laboratory safety tests, vital signs, ECG results, physical examination findings, and any other parameter relevant for safety assessment.

The PK population consists of all participants who receive at least 1 dose of IP and provide at least 1 quantifiable PK plasma sample. This population will be used to summarize PK concentrations and parameters over time. Pharmacokinetic parameter estimates will be calculated by a standard noncompartmental method of analysis; these may include (but are not limited to) measures of the extent of absorption using estimates of the area under the plasma concentration-time curve (AUC), the maximum observed drug concentration (Cmax), and the time to reach maximum drug concentration (Tmax). Other parameters may be included at the discretion of the pharmacokineticist and as data permit.

### Method of Assigning Participants to Treatment Groups:

In this parallel-group randomized study, participants who meet study entry criteria will be randomly assigned in a 1:1:1:1:1 ratio, by history of hysterectomy, to 1 of the 5 treatment groups indicated in Table 2. The randomization schedule will be computer-generated using a permuted block algorithm and will randomly allocate IP to randomization numbers. Study center will not be a blocking factor in the randomization schedule. The randomization numbers will be assigned sequentially by a member of the ICON Clinical Research Team who is not otherwise involved in the study. No one involved in the study performance will have access to the randomization schedule before official unblinding of treatment assignment. No participant will be randomized into this study more than once.

The different tamoxifen-dose vaginal inserts and placebo are indistinguishable by appearance, smell, and feel. All participants, investigators, and study personnel involved in the conduct of the study, including data management, will be blinded to treatment assignment with the exception of a specified unblinded statistician from ICON Clinical Research who will have access to the randomization code. The unblinded study personnel will not participate in study procedures or data analysis prior to unblinding of the study data to all study-related personnel. Overall unblinding will take place at the end of the study only after database lock has been achieved.

### Statistical Analysis:

An analysis of covariance (ANCOVA) model will be used to assess treatment differences in change from baseline for vaginal pH, vaginal cytology, dyspareunia, and most bothersome symptom, using baseline score as a covariate. Summary statistics will be provided for the variables described below. For continuous variables, these statistics will typically include the number of participants, mean, standard deviation (SD), median, minimum, and maximum. For categorical variables, these statistics will typically include the number and percentage of participants in each category. The denominator for percentage will be based on the number of participants appropriate for the purpose of analysis. For all calculations of change from baseline, the last observation recorded before the first administration of the insert (tamoxifen or placebo) will be considered as the baseline observation.

### Secondary Analyses

The secondary efficacy endpoints are the following:
Change of vaginal pH from baseline to Day 57
Change in vaginal cytology (maturation index; percentage of basal and superficial cells) from baseline to Day 57
Change in most bothersome symptom from baseline to Day 56
Change in severity of dyspareunia from baseline to Day 56
Correlation between exposure and local changes
   Descriptive summaries (mean, SD, median, minimum, and maximum) involving change of vaginal pH and vaginal cytology will be presented by treatment group at each time point. In addition, an ANCOVA model will be used to assess treatment differences in change from baseline for vaginal pH, vaginal cytology, dyspareunia, and most bothersome symptom, using baseline score as a covariate.

### Example 2: Pharmacokinetics, Safety and Preliminary Pharmacodynamic Evaluation of DARE-VVA1: A Soft Gelatin Capsule Containing Tamoxifen for the Treatment of Vulvovaginal Atrophy

This study aimed to measure safety, systemic pharmacokinetics and preliminary efficacy of a vaginal tamoxifen capsule (DARE-VVA1) among postmenopausal women with moderate-to severe vulvovaginal atrophy. DARE-VVA1 was safe and resulted in minimal systemic exposure to tamoxifen.

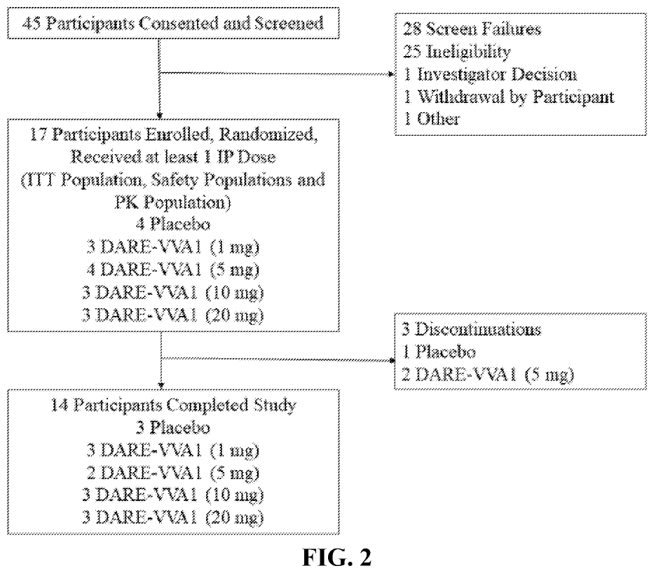

As shown in FIG. 2, 45 participants were screened for the study and 28 participants were screen failures. Therefore 17 women were enrolled, all of whom had an intact uterus and were randomized and dosed with at least one dose, IP. A total of 14 women completed the study. The three participants who discontinued the study did so due to protocol deviations (n=1) and non-compliance with the study medication (n=2).

Table 7 presents the demographics of the intent-to-treat population. All enrolled women had a normal screening thyroid stimulating hormone (TSH) concentration, normal Pap smear results and a normal screening mammogram. All participants were menopausal based on the presence of spontaneous amenorrhea for 12 months or longer.

TABLE 7
Demographics of the intent-to-treat population

| | | DARE-VVA1 | DARE-VVA1 | DARE-VVA1 | DARE-VVA1 | |
|---|---|---|---|---|---|---|
| | | 1 mg | 5 mg | 10 mg | 20 mg | Overall |
| Characteristic | Placebo (N = 4) | (N = 3) | (N = 4) | (N = 3) | (N = 3) | (N = 17) |
| Age (years) | | | | | | |
| Mean (SD) | 61.3 (5.38) | 65.7 (1.33) | 59.3 (2.99) | 57.7 (8.08) | 61.3 (4.16) | 60.8 (4.97) |
| Median (min, max) | 60.5 (56, 68) | 66.0 (64, 67) | 59.0 (56, 63) | 58.0 (49, 65) | 60.0 (68, 60) | 60.0 (49, 68) |
| Race, n (%) | | | | | | |
| White | 4 (100.0) | 3 (100.0) | 4 (100.0) | 3 (100.0) | 3 (100.0) | 17 (100.0) |
| Not Hispanic or Latino | 4 (100.0) | 3 (100.0) | 4 (100.0) | 3 (100.0) | 3 (100.0) | 17 (100.0) |
| Height (feet) | | | | | | |
| Mean (SD) | 5.31 (0.22) | 5.55 (0.19) | 5.52 (0.12) | 5.33 (0..18) | 5.35 (0.18) | 5.41 (0.19) |
| Median (min, max) | 5.24 (5.13, 5.55) | 5.64 (5.33, 5.61) | 5.51 (5.41, 5.59) | 3.42 (6.12, 5.40) | 5.43 (5.13, 5.42) | 5.43 (5.12, 5.61) |
| Weight (lbs) | | | | | | |
| Mean (SD) | 146.15 (42.64) | 164.49 (35.34) | 166.26 (36.13) | 143.55 (13.37) | 152.48 (31.16) | 146.15 (42.64) |
| Median (min, max) | 131.97 | 144.43 | 159.97 | 136.08 | 143.99 | 131.97 |
| | (112.23, 208.37) | (143.99, 205.07) | (130.54, 214.55) | (135.61, 158.98) | (112.23, 214.55) | (112.23, 208.37) |
| BMI (kg/m²) | | | | | | |
| Mean (SD) | 25.03 (5.29) | 26.03 (4.87) | 26.58 (5.49) | 24.67 (2.22) | 23.70 (3.68) | 25.27 (4.15) |
| Median (min, max) | 24.20 (19.5, 32.2) | 24.80 (21.9, 31.4) | 25.60 (21.6, 33.5) | 25.30 (22.2, 26.5) | 22.60 (20.7, 27.8) | 24.70 (19.5, 33.5) |
| Screening TSH (uIU/ml) | | | | | | |
| Mean (SD) | 1.98 (0.63) | 1.19 (0.26) | 2.52 (0.67) | 2.55 (0.68) | 1.39 (0.74) | 1.96 (0.78) |
| Median (min, max) | 2.18 (1.1, 2.5) | 1.19 (0.9, 1.4) | 2.77 (1.6, 3.0) | 2.94 (1.8, 2.9) | 1.36 (0.6, 2.0) | 2.02 (0.6, 3.0) |

BMI, body mass index;
max, maximum;
min, minimum;
SD, standard deviation;
TSH, thyroid stimulating hormone.

### Safety Data

Table 8 presents the TEAE data and Table 8A presents the total exposure to tamoxifen (mg), number of days of dosing, protocol adherence per participant self-reported diary entries and the system organ preferred term for product-related TEAEs. As already noted, 14 of the 17 randomized participants completed the entire 56-day treatment period. The majority of participants were greater than 95% compliant with the dosing regimen, per their diary report.

The were no severe adverse events and no TEAEs which led to study product or study discontinuation. The majority of participants who received at least one dose of the study IP (15/17, 88.2%) reported at least one TEAE. Of the 72 reported TEAEs for all participants, the highest number of reported TEAEs occurred in the 20 mg DARE-VVA1 dosing group (n=20 TEAEs), with the second highest reported by the placebo group (n=15 total reported TEAEs). All TEAEs were mild or moderate in severity.

Almost half (8/17, 47%) of the participants reported at least one TEAE which was deemed as possibly related or related to study IP use (Table 8 and Table 8A). Of the 22 TEAEs reported by the 17 participants that were deemed possibly related or related to study IP use, the highest number (n=8) was reported by the placebo users (Table 8). Finally, 5/17 (29%) participants reported at least one ADR, with a total of nine ADRs being reported in the study. There were no unexpected ADRs reported in this study.

Of the 15 participants who reported at least one TEAE, nine reported a TEAE related to the reproductive system, with vulvovaginal discomfort (n=5 participant reports) and vulvovaginal pruritus (n=4 participant reports) being the most common organ system preferred term (see Table 8A).

The mean local erythema scores at all visits, for all dosing groups, were in the none/absent (score 0) to mild (score 1) range, with a few outliers in the moderate (score 2) grading, with no discernible pattern or correlation to group. All local edema scores at all visits, for all dosing groups, were graded as none (score 0) or mild (score 1). All endometrial stripe measurements were normal at baseline and at 57 days of treatment, with the maximum measurement not exceeding 4.0 mm. All vaginal speculum examinations were reported as normal or had an abnormality or finding that was deemed not clinically significant by the investigator.

Finally, there were isolated, not clinically significant changes in serum chemistry, hematology and coagulation parameters and concentrations, and urinalyses, with no discernible pattern or dose relationship. There were no clinically significant electrocardiogram findings or changes from baseline during the study.

TABLE 8
Safety Data

| | Placebo | DARE-VVA1 | DARE-VVA1 | DARE-VVA1 | DARE-VVA1 | Overall |
|---|---|---|---|---|---|---|
| | (N = 4) | 1 mg (N = 3) | 5 mg (N = 4) | 10 mg (N = 3) | 20 mg (N = 3) | (N = 17) |
| Treatment emergent adverse events | | | | | | |
| Number of women (% of group) | 3 (75%) | 2 (67%) | 4 (100%) | 3 (100%) | 3 (100%) | 15 (88.2%) |
| reporting at least one TEAE | | | | | | |
| Total number of TEAEs | 15 | 10 | 13 | 14 | 20 | 72 |
| reported per group | | | | | | |
| Number of women (% of group) | 2 (50%) | 1 (33%) | 2 (50%) | 1 (33%) | 2 (67%) | 8 (47%) |
| reporting at least one TEAE with | | | | | | |
| causality possibly related | | | | | | |
| or related to IP | | | | | | |
| Total number of TEAEs with | 8 | 2 | 5 | 4 | 5 | 22 |
| causality possibly related or | | | | | | |
| related to IP* | | | | | | |
| Number of women (% of group) | 2 (50%) | 0 (0%) | 2 (50%) | 1 (33%) | 0 (0%) | 5 (29%) |
| reporting at least one adverse | | | | | | |
| drug reaction | | | | | | |
| Total number of adverse drug | 3 | 0 | 2 | 4 | 0 | 9 |
| reactions reported per group | | | | | | |
| Total number of TEAEs per | 0 | 0 | 0 | 0 | 0 | 0 |
| group of Grade 3 or higher | | | | | | |
| Total TEAEs leading to IP or | 0 | 0 | 0 | 0 | 0 | 0 |
| study discontinuation | | | | | | |
| Local vulvovaginal imitation | | | | | | |
| scores (0, 1, 2, 3 for none, | | | | | | |
| mild, moderate, severe, respectively) | | | | | | |
| Erythema | | | | | | |
| Baseline (mean, SD) | 0.8 (0.96) | 0.3 (0.58) | 0.0 (0.0) | 0.0 (0.0) | 0.3 (0.58) | 0.3 (0.59) |
| Day 18 (mean, SD) | 0.5 (0.58) | 0.7 (0.58) | 0.0 (0.0) | 1.3 (1.15) | 0.7 (0.58) | 0.6 (0.72) |
| Day 28 (mean, SD) | 0.3 (0.50) | 0.3 (0.58) | 0.7 (0.58) | 0.0 (0.0) | 0.3 (0.58) | 0.3 (0.48) |
| Day 42 (mean, SD) | 0.5 (0.71) | 0.7 (1.15) | 0.0 (0.0) | 0.0 (0.0) | 1.0 (1.41) | 0.4 (0.77) |
| Day 57 (mean, SD) | 0.0 (0.0) | 0.3 (0.56) | 0.0 (0.0) | 0.0 (0.0) | 0.3 (0.58) | 0.1 (0.36) |
| Day 63 (mean, SD) | 0.0 (0.0) | 0.7 (1.15) | 0.5 (0.71) | 0.3 (0.58) | 0.0 (0.0) | 0.3 (0.61) |
| Edema | | | | | | |
| Baseline (mean, SD) | 0.5 (0.58) | 0.0 (0.0) | 0.0 (0.0) | 0.0 (0.0) | 0.3 (0.58) | 0.2 (0.39) |
| Day 18 (mean, SD) | 0.0 (0.0) | 0.3 (0.58) | 0.0 (0.0) | 0.0 (0.0) | 0.3 (0.58) | 0.1 (0.34) |
| Day 28 (mean, SD) | 0.3 (0.50) | 0.0 (0.0) | 0.3 (0.58) | 0.0 (0.0) | 0.3 (0.58) | 0.2 (0.48) |
| Day 42 (mean, SD) | 0.0 (0.0) | 0.0 (0.0) | 0.0 (0.0) | 0.0 (0.0) | 0.5 (0.71) | 0.1 (0.28) |
| Day 57 (mean, SD) | 0.0 (0.0) | 0.3 (0.58) | 0.5 (0.71) | 0.0 (0.0) | 0.7 (0.58) | 0.3 (0.47) |
| Day 63 (mean, SD) | 0.0 (0.0) | 0.3 (0.58) | 0.0 (0.0) | 0.0 (0.0) | 0.0 (0.0) | 0.1 (0.27) |
| Transvaginal ultrasound | | | | | | |
| endometrial stripe thickness (mm) | | | | | | |
| Baseline (mean, SD) | 1.88 (0.63) | 1.80 (1.06) | 3.25 (0.96) | 2.00 (1.00) | 2.20 (1.59) | 2.26 (1.08) |
| Day 57 (mean, SD) | 1.83 (0.76) | 2.00 (1.00) | 3.50 (0.71) | 2.67 (0.58) | 2.73 (2.00) | 2.48 (1.13) |

*System Organ Class Preferred Term of these TEAEs presented in Table 8A
IP, investigational product; SD, Standard deviation; TEAE, treatment emergent adverse event.

TABLE 8A
TEAE System Organ Class Preferred Term, Exposure, Adherence and Participant
Reported Compliance Data by Dosing Group

| | | | | | | |
|---|---|---|---|---|---|---|
| | | DARE- | DARE- | DARE- | DARE- | |
| | | VVA1 | VVA1 | VVA1 | VVA1 | |
| | Placebo | 1 mg | 5 mg | 10 mg | 20 mg | Overall |
| Variable | N = 4 | N = 3 | N = 4 | N = 3 | N = 3 | N = 17 |

Participant's Total Dose (mg) (number of insertions X dose assigned)

| | | | | | | |
|---|---|---|---|---|---|---|
| Mean (SD) | 0.0 (0.0) | 25.7 (0.58) | 117.5 | 260.0 (0.0) | 520.0 (0.0) | 169.8 |
| | | | (21.79) | | | (190.78) |
| Median (Min, | 0.0 (0, 0) | 26.0 (25, 26) | 127.5 (85, 130) | 260.0 (260, 260) | 520.0 (520, 520) | 125.0 (0, 520) |
| Max) | | | | | | |

Duration of Exposure (days) (date of last day of treatment-date of first day of treatment + 1)

| | | | | | | |
|---|---|---|---|---|---|---|
| Mean (SD) | 49.3 | 56.7 (1.15) | 42.8 | 56.0 (0.0) | 56.0 (0.0) | 51.4 |
| | (14.17) | | (18.39) | | | (11.59) |
| Median (Min, | 56.0 (28, | 56.0 (56, | 49.0 (17, | 56.0 (56, | 56.0 (56, | 56.0 (17, |
| Max) | 57) | 58) | 56) | 56) | 56) | 58) |

Treatment Compliance (100% X (number of days the study treatment is in place correctly/26) ^{a}

| | | | | | | |
|---|---|---|---|---|---|---|
| Mean (SD) | 101.0 | 98.7 (2.22) | 89.4 | 97.4 (2.22) | 100.0 (0) | 97.1 (8.76) |
| | (4.84) | | (16.43) | | | |
| Median (Min, | 100.0 (96, | 100.0 (96, | 96.2 (65, | 96.2 (96, | 100.0 (100, | 100.0 (65, |
| Max) | 108) | 100) | 100) | 100) | 100) | 108) |

Number of TEAEs per Dosing Group with Causality of Possibly Related or Related to Study Product:
System Organ Class Preferred Term

| | | | | | | |
|---|---|---|---|---|---|---|
| Reproductive | | | | | | |
| system disorders | | | | | | |
| Overall | 2 | 1 | 2 | 1 | 2 | 8 |
| Reproductive | | | | | | |
| system disorders | | | | | | |
| Vulvovaginal | 1 | 0 | 1 | 0 | 1 | 3 |
| Discomfort or | | | | | | |
| Irritation | | | | | | |
| Vulvovaginal | 1 | 0 | 0 | 0 | 1 | 2 |
| Pruritus | | | | | | |
| Genital Rash | 0 | 1 | 0 | 0 | 0 | 1 |
| Postmenopausal | 0 | 0 | 1 | 0 | 0 | 1 |
| Bleeding^{b} | | | | | | |
| Vulvovaginal | 0 | 0 | 0 | 1 | 0 | 1 |
| Erythema | | | | | | |
| Nervous System | 6 | 1 | 1 | 0 | 0 | 8 |
| Disorders (e.g. | | | | | | |
| Headache, | | | | | | |
| Dizziness) | | | | | | |
| Renal and | 0 | 0 | 0 | 0 | 1 | 1 |
| Urinary | | | | | | |
| Disorders | | | | | | |
| (Hematuria) | | | | | | |
| Vascular | 0 | 0 | 1 | 0 | 0 | 1 |
| Disorders (e.g. | | | | | | |
| hot flush, | | | | | | |
| hypertension) | | | | | | |
| Gastrointestinal | 0 | 0 | 1 | 0 | 0 | 1 |
| Disorders (e.g. | | | | | | |
| Nausea, Rectal | | | | | | |
| bleeding) | | | | | | |
| General | 0 | 0 | 0 | 3 | 0 | 3 |
| disorders and | | | | | | |
| administration | | | | | | |
| site disorders | | | | | | |
| (e.g. catheter site | | | | | | |
| pain, burning | | | | | | |
| sensation with IP | | | | | | |
| insertion) | | | | | | |
| TOTALS | 8 | 2 | 4 | 4 | 3 | 22 |

^{a} = Compliance data was >100% for women who used more than 26 doses, due to appointment scheduling logistics.
^{b} = Subsequent Endometrial Biopsy Benign

### Systemic Plasma Pharmacokinetics

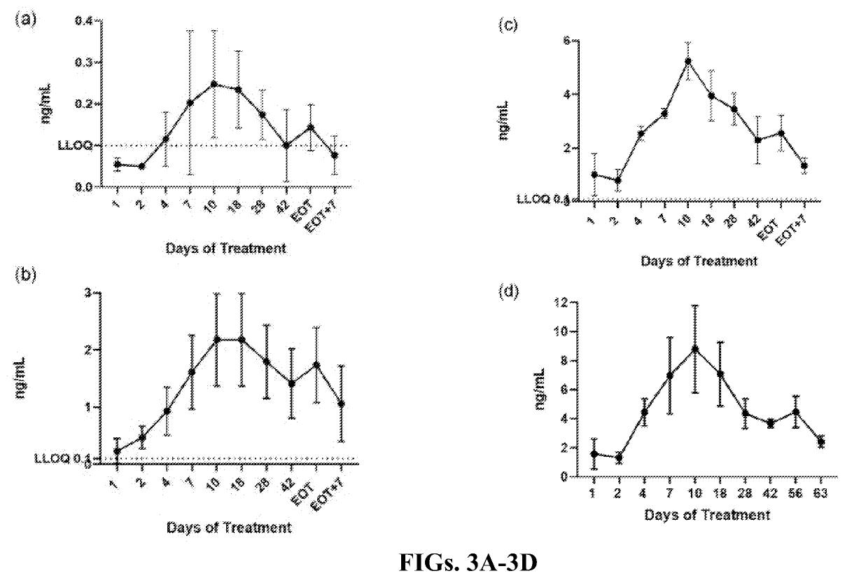

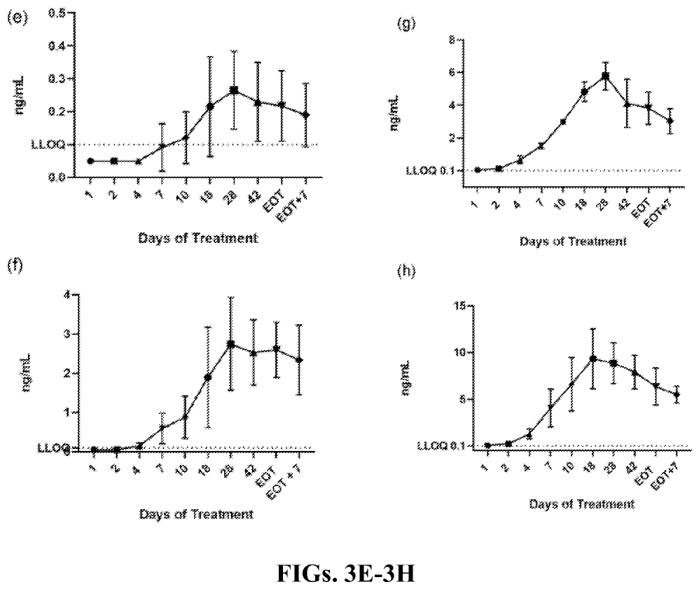

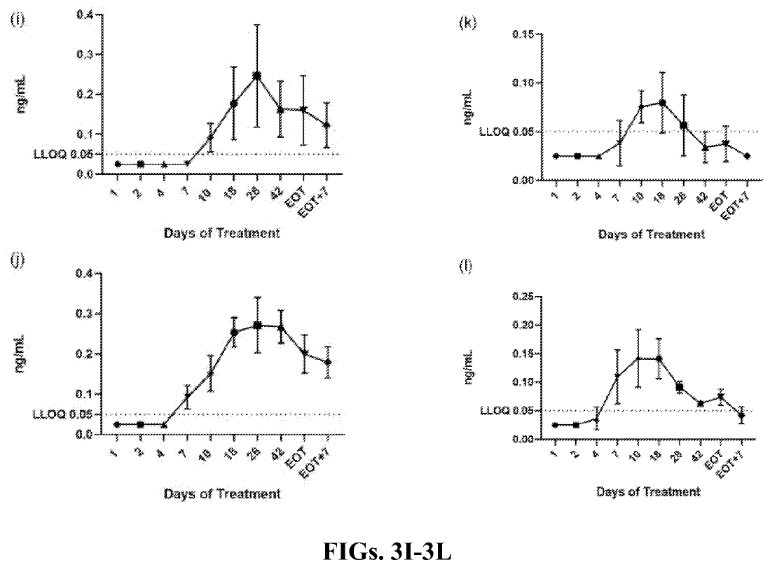

PK parameters for tamoxifen and NDT are presented in Table 9A, and FIG. 3A-3L shows the mean (standard deviation) plasma concentrations by time for tamoxifen and the three metabolites (ng/ml). As expected, the highest median plasma concentrations of tamoxifen occurred in the 20 mg dosing group. The highest median plasma tamoxifen concentration measured in the study was 8.29 ng/ml, measured on day 10 of dosing in the 20 mg dosing group. The single maximum plasma tamoxifen concentration observed in the study was 12.0 ng/ml, from a participant randomized to the 20 mg dose at the same time point (see Table 9A).

The major metabolite of orally dosed tamoxifen is NDT. As with the tamoxifen plasma PK profile, VVA1 20 mg users had the highest concentrations of NDT. The highest median plasma concentration of NDT was 8.43 ng/ml, measured on day 18 of dosing, with a single maximum plasma concentration of NDT recorded at 12.9 ng/ml on the same day. The mean and median PK parameters of plasma tamoxifen and NDT are presented in Table 9.

For endoxifen concentrations, DARE-VVA1 1 mg users had BLQ concentrations of this metabolite at all sampling points. DARE-VVA1 5 mg users had BLQ concentrations of this metabolite at all time points except day 42 where median endoxifen concentrations were 0.056 ng/ml (range 0.03-0.14 ng/ml). Table 9A and FIGS. 3I and 3J demonstrated that DARE-VVA1 10 mg users had BLQ concentrations of plasma endoxifen until day 10 of dosing, while DARE-VVA1 20 mg users had BLQ concentrations of plasma endoxifen until day 7 of dosing.

DARE-VVA1 1 mg and 5 mg users had concentrations of the metabolite 4-OHT that were BLQ at all time points measured. As shown in Table 9A and FIG. 3K, DARE-VVA1 10 mg users had BLQ concentrations of this metabolite until day 10 of dosing, and plasma 4-OHT concentrations were then low but measurable between days 10 and 42, when concentrations returned to BLQ levels. As shown in Table 9A and FIG. 3L, DARE-VVA1 20 mg users had BLQ concentrations of 4-OHT until day 7 of dosing and had very low concentrations of this metabolite throughout the observation period.

TABLE 9
Pharmacokinetic parameters for plasma tamoxifen and N-desmethyl tamoxifen (NDT).

| | | | | | |
|---|---|---|---|---|---|
| | | DARE-VVA1 | DARE-VVA1 | DARE-VVA1 | DARE-VVA1 |
| Parameter | Statistic | 1 mg (n = 3) | 5 mg (n = 4) | 10 mg (n = 3) | 20 mg (n = 3) |

Tamoxifen

| | | | | | |
|---|---|---|---|---|---|
| Day 1 Cₘₐₓ (ng/ml) | Mean | 0.04 | 0.49 | 2.08 | 2.66 |
| | SD | 0.06 | 0.20 | 0.51 | 0.85 |
| | Median | 0 | 0.57 | 2.17 | 2.97 |
| Day 1 Tₘₐₓ (h) | Median | 4.98 | 15.96 | 2.00 | 2.43 |
| | Minimum | 4.98 | 5.00 | 2.00 | 2.02 |
| | Maximum | 4.98 | 24.08 | 2.00 | 4.00 |
| Day 1 AUC_{0-24 h} (h*ng/ml) | Mean | 0 | 9.22 | 25.05 | 39.08 |
| | SD | 0 | 4.44 | 11.89 | 12.27 |
| | Median | 0 | 10.84 | 20.26 | 44.60 |
| Day 56 Cₘₐₓ (ng/ml) | Mean | 0.19 | 2.03 | 3.307 | 5.69 |
| | SD | 0.06 | 0.87 | 0.38 | 1.87 |
| | Median | 0.17 | 2.03 | 3.47 | [symbol] |
| Day 56 Tₘₐₓ (h) | Median | 4.00 | 6.51 | 8.00 | 8.00 |
| | Minimum | 0.50 | 5.02 | 4.00 | 8.00 |
| | Maximum | 8.05 | 8.00 | 12.00 | 12.00 |
| Day 56 AUC [symbol] (h*ng/ml) | Mean | 3.55 | 45.17 | 68.61 | 113.00 |
| | SD | 1.09 | 22.09 | 8.21 | 21.81 |
| | Median | 3.25 | 45.17 | 70.71 | 102.00 |
| Day 56 T_{1/2} (h) | Mean | | 182.21 | 109.29 | 190.80 |
| | SD | | 16.70 | | |
| | Median | | 182.21 | 109.29 | 190.80 |

NDT

| | | | | | |
|---|---|---|---|---|---|
| Day 1 Cₘₐₓ (ng/ml) | Mean | 0 | 0 | 0.09 | 0.20 |
| | SD | 0 | 0 | 0.16 | 0.17 |
| | Median | 0 | 0 | 0 | 0.30 |
| Day 1 Tₘₐₓ (hours) | Median | | | 24.07 | 24.02 |
| | Minimum | | | 24.07 | 24.00 |
| | Maximum | | | 24.07 | 24.03 |
| Day 1 AUC_{0-24 h} (h*ng/ml) | Mean | 0 | 0 | 1.01 | 1.74 |
| | SD | 0 | 0 | 1.74 | 2.47 |
| | Median | 0 | 0 | 0 | 1.74 |
| Day 56 Cₘₐₓ (ng/ml) | Mean | 0.27 | 2.87 | 4.49 | 8.13 |
| | SD | 0.13 | 0.93 | 1.45 | 2.90 |
| | Median | 0.20 | 2.87 | 5.12 | [symbol] |
| Day 56 Tₘₐₓ (h) | Median | 5.00 | 24.01 | 8.00 | 0.50 |
| | Minimum | 0.50 | 24.00 | 0.50 | 0.50 |
| | Maximum | 8.05 | 24.02 | 24.02 | 0.50 |
| Day 56 AUC [symbol] (h*ng/ml) | Mean | 4.85 | 63.65 | 91.91 | 141.9 |
| | SD | 2.50 | 24.18 | 24.33 | 39.06 |
| | Median | 3.54 | 63.65 | 99.82 | 134.20 |

AUC, area under the plasma concentration-time curve; Cₘₐₓ, maximum observed drug concentration; SD, standard deviation; Tₘₐₓ, maximum drug concentration; T_{1/2}, half life. [symbol]
indicates data missing or illegible when filed

TABLE 9A
Plasma concentrations of tamoxifen and metabolites based on dosing group and
sampling time

| Plasma Analyte & Dosing Group | | | | | | | | | | | | | |
|---|---|---|---|---|---|---|---|---|---|---|---|---|---|
| Days of Dosing (Trough Values for all days except Day 1) | | | | | | | | | | Post Day 56 Dosing | | | |
| Day of Dosing | | | | | | | | | | | | | |
| 1 | | 2 | 4 | 7 | 10 | 18 | 28 | 42 | 56 | 56 | 56 | 57 | 63 |
| Hours Post Dosing | | | | | | | | | | | | | |
| 8 | 12 | | | | | | | | | 8 | 12 | 24 | 7 |
| hrs | hrs | | | | | | | | | hrs | hrs. | hrs. | days |

Tamoxifen (ng/mL)
DARE-VVA1 (1 mg)

| | | | | | | | | | | | | |
|---|---|---|---|---|---|---|---|---|---|---|---|---|
| Median | BLQ | 0.12 | 0.17 | 0.22 | 0.23 | 0.15 | BLQ | 0.11 | 0.14 | 0.15 | 0.13 | BLQ |
| Geo. | | 0.10 | 0.15 | 0.23 | 0.22 | 0.17 | | 0.11 | 0.12 | 0.13 | 0.14 | |
| Mean | | | | | | | | | | | | |
| CV | | 72.76 | 138.19 | 56.75 | 44.06 | 32.76 | | 83.54 | 99.07 | 23.45 | 38.33 | |
| Geo. | | | | | | | | | | | | |
| Mean | | | | | | | | | | | | |

DARE-VVA1 (5 mg)

| | | | | | | | | | | | | | | |
|---|---|---|---|---|---|---|---|---|---|---|---|---|---|---|
| Median | 0.54 | 0.49 | 0.54 | 1.05 | 1.64 | 2.31 | 2.39 | 1.59 | 1.12 | 1.37 | 1.99 | 1.88 | 1.98 | 1.06 |
| Geo. | 0.38 | 0.38 | 0.43 | 0.82 | 1.51 | 2.05 | 2.03 | 1.72 | 1.34 | 1.29 | 1.90 | 1.73 | 1.87 | 0.95 |
| Mean | | | | | | | | | | | | | | |
| CV | 82.69 | 72.59 | 59.80 | 70.39 | 43.99 | 44.05 | 48.90 | 35.42 | 41.53 | 52.39 | 44.43 | 63.97 | 52.11 | 75.14 |
| Geo | | | | | | | | | | | | | | |
| Mean | | | | | | | | | | | | | | |

DARE-VVA1 10 mg

| | | | | | | | | | | | | | | |
|---|---|---|---|---|---|---|---|---|---|---|---|---|---|---|
| Median | 0.94 | 0.67 | 0.58 | 2.57 | 3.32 | 4.87 | 4.19 | 3.72 | 2.45 | 1.930 | 2.87 | 2.93 | 2.92 | 1.42 |
| Geo. | 1.11 | 0.82 | 0.74 | 2.54 | 3.28 | 5.21 | 3.87 | 3.41 | 2.17 | 1.87 | 2.91 | 3.06 | 2.92 | 1.33 |
| Mean | | | | | | | | | | | | | | |
| CV | 61.48 | 57.03 | 49.19 | 10.58 | 5.60 | 12.92 | 25.58 | 18.25 | 45.2 | 29.85 | 4.12 | 10.94 | 16.32 | 22.81 |
| Geo | | | | | | | | | | | | | | |
| Mean | | | | | | | | | | | | | | |

DARE-VVA1 20 mg

| | | | | | | | | | | | | | | |
|---|---|---|---|---|---|---|---|---|---|---|---|---|---|---|
| Median | 2.40 | 1.45 | 1.45 | 4.94 | 6.80 | 8.29 | 6.83 | 3.88 | 3.68 | 3.95 | 4.63 | 4.58 | 4.36 | 2.39 |
| Geo. | 1.95 | 1.38 | 1.27 | 4.36 | 6.62 | 8.46 | 6.85 | 4.28 | 3.67 | 4.03 | 5.42 | 4.62 | 4.82 | 2.40 |
| Mean | | | | | | | | | | | | | | |
| CV | 45.07 | 41.39 | 33.63 | 23.21 | 40.95 | 35.05 | 32.04 | 22.72 | 7.91 | 15.74 | 33.14 | 15.80 | 18.10 | 16.48 |
| Geo | | | | | | | | | | | | | | |
| Mean | | | | | | | | | | | | | | |

N-desmethyl tamoxifen (NDT)
DARE-VVAI (1 mg)

| Median | BLQ | 0.11 | 0.25 | 0.26 | 0.19 | 0.18 | 0.17 | 0.16 | 0.15 | 0.15 |
|---|---|---|---|---|---|---|---|---|---|---|
| Geo. | | 0.10 | 0.16 | 0.25 | 0.21 | 0.20 | 0.19 | 0.17 | 0.19 | 0.17 |
| Mean | | | | | | | | | | |
| CV | | 80.76 | 138.86 | 50.25 | 53.27 | 65.81 | 81.28 | 34.33 | 54.57 | 51.29 |
| Geo | | | | | | | | | | |
| Mean | | | | | | | | | | |

DARE-VVA1 (5 mg)

| Median | BLQ | 0.18 | 0.50 | 0.87 | 1.81 | 3.30 | 2.45 | 2.38 | 2.78 | 2.52 | 2.87 | 2.34 |
|---|---|---|---|---|---|---|---|---|---|---|---|---|
| Geo. | | 0.13 | 0.51 | 0.75 | 1.54 | 2.54 | 2.44 | 2.34 | 2.71 | 2.38 | 2.79 | 2.25 |
| Mean | | | | | | | | | | | | |
| CV | | 74.81 | 71.65 | 76.31 | 91.68 | 55.91 | 34.46 | 26.01 | 32.35 | 50.36 | 34.04 | 40.31 |
| Geo | | | | | | | | | | | | |
| Mean | | | | | | | | | | | | |

DARE-VVA1 10 mg

| Median | BLQ | 0.55 | 1.44 | 3.03 | 5.05 | 6.19 | 4.08 | 3.84 | 3.88 | 4.00 | 4.24 | 2.90 |
|---|---|---|---|---|---|---|---|---|---|---|---|---|
| Geo. | | 0.64 | 1.51 | 2.99 | 4.80 | 5.74 | 3.94 | 3.35 | 3.57 | 3.76 | 3.81 | 2.97 |
| Mean | | | | | | | | | | | | |
| CV | | 35.64 | 10.86 | 4.02 | 12.87 | 15.39 | 38.89 | 34.83 | 20.27 | 26.56 | 37.26 | 25.30 |
| Geo | | | | | | | | | | | | |
| Mean | | | | | | | | | | | | |

DARE-VVA1 20 mg

| | | | | | | | | | | | | | | |
|---|---|---|---|---|---|---|---|---|---|---|---|---|---|---|
| Median | BLQ | 0.11 | 0.30 | 1.31 | 4.54 | 7.14 | 8.43 | 8.47 | 7.91 | 7.46 | 5.26 | 5.56 | 5.85 | 5.04 |
| Geo. | | 0.10 | 0.16 | 1.21 | 3.66 | 6.15 | 8.99 | 8.68 | 7.80 | 6.83 | 6.35 | 5.25 | 5.87 | 5.45 |
| Mean | | | | | | | | | | | | | | |
| CV | | 67.02 | 136.73 | 48.80 | 66.39 | 52.26 | 34.25 | 24.68 | 23.13 | 28.99 | 39.52 | 26.87 | 24.34 | 15.48 |
| Geo | | | | | | | | | | | | | | |
| Mean | | | | | | | | | | | | | | |

4-hydroxy tamoxifen (4-OHT) (ng/mL)
DARE-VVA1 10 mg

| Median | BLQ | 0.08 | 0.07 | 0.06 | BLQ |
|---|---|---|---|---|---|
| Geo. Mean | | 0.07 | 0.08 | 0.05 | |
| CV | | 23.29 | 38.37 | 70.69 | |
| Geo | | | | | |
| Mean | | | | | |

DARE-VVA1 20 mg

| Median | BLQ | 0.13 | 0.16 | 0.14 | 0.10 | 0.06 | 0.08 | 0.06 | 0.06 | 0.08 | 0.05 |
|---|---|---|---|---|---|---|---|---|---|---|---|
| Geo | | 0.10 | 0.13 | 0.14 | 0.09 | 0.06 | 0.08 | 0.07 | 0.07 | 0.07 | 0.04 |
| Mean | | | | | | | | | | | |
| CV | | 55.45 | 41.90 | 25.83 | 11.49 | 4.83 | 29.80 | 29.77 | 14.35 | 15.95 | 42.53 |
| Geo | | | | | | | | | | | |
| Mean | | | | | | | | | | | |

N-desmethyl-4-hydroxytamoxifen (Endoxifen) (ng/mL)
DARE-VVA1 10 mg

| Median | BLQ | 0.10 | 0.17 | 0.24 | 0.15 | 0.10 | 0.12 | 0.12 | 0.11 | 0.09 |
|---|---|---|---|---|---|---|---|---|---|---|
| Geo. | | 0.09 | 0.16 | 0.22 | 0.15 | 0.13 | 0.14 | 0.15 | 0.14 | 0.12 |
| Mean | | | | | | | | | | |
| CV | | 47.61 | 61.31 | 61.81 | 44.92 | 58.39 | 51.26 | 47.92 | 57.70 | 43.77 |
| Geo | | | | | | | | | | |
| Mean | | | | | | | | | | |

DARE-VVA1 20 mg

| Median | BLQ | 0.11 | 0.18 | 0.25 | 0.26 | 0.27 | 0.22 | 0.21 | 0.18 | 0.18 | 0.18 |
|---|---|---|---|---|---|---|---|---|---|---|---|
| Geo. | | 0.09 | 0.15 | 0.25 | 0.27 | 0.27 | 0.22 | 0.19 | 0.18 | 0.18 | 0.18 |
| Mean | | | | | | | | | | | |
| CV | | 36.51 | 33.26 | 14.52 | 25.36 | 15.21 | 18.13 | 34.56 | 10.63 | 23.55 | 22.54 |
| Geo | | | | | | | | | | | |
| Mean | | | | | | | | | | | |

### Vaginal pH and Vaginal Maturation Index

Both of these preliminary assessments of product efficacy were secondary endpoints, as the sample size was not powered to demonstrate significant differences for individual dosing groups. When all active DARE-VVA1 doses were combined into one group, there was a significant decrease in vaginal pH between baseline and end of treatment (day 56), with the DARE-VVA1 20 mg cohort experiencing the largest decrease in median vaginal pH (−0.7) (Table 4). The 1 mg and 5 mg dosing cohorts experienced a median decrease in vaginal pH of −0.4 and 0, respectively.

Similar to the data for vaginal pH, the overall VMI increased with treatment, although not significantly so for any individual dosing group (all p>0.09) (Table 10). The largest increase in total VMI, reflecting the largest shift to healthy, superficial cells, occurred in the 20 mg VVA1 dosing group. This dosing group also had the largest increase in superficial cells, from a median of 0% at baseline to a median of 54% at the end of treatment (p=0.06). There was a statistically significant decrease in vaginal parabasal cells, from a median of 75% at baseline to a median of 1% at end of treatment (p=0.04), for all active study product users (Table 10).

TABLE 10
Vaginal pH and vaginal cytology indices at baseline and end of treatment by dosing group

| | Baseline pre-treatment (day 1) | | | Day 56 or 57 (end of treatment) | | | |
|---|---|---|---|---|---|---|---|
| Parameter | Median | Mean | SD | Median | Mean | SD | p-Value |
| Vaginal pH | | | | | | | |
| Placebo (n = 4) | 5.3 | 5.6 | 0.6 | 5.3 | 5.4 | 0.51 | 1.00 |
| DARE-VVA1 1 mg | 5.7 | 5.7 | 0.35 | 5.3 | S.3 | 0.65 | 0.35 |
| DARE-VVA1 5 mg | 5.2 | 5.3 | 0.33 | 5.2 | 5.2 | 0.71 | 0.43 |
| DARE-VVA1 100 mg | 5.3 | 5.5 | 0.40 | 5.0 | 5.2 | 0.40 | 0.69 |
| DARE-VVA1 20 mg | 5.7 | 6.0 | 0.89 | 5.0 | 5.1 | 0.17 | 0.72 |
| DARE VVA1 all actives | 5.3 | 5.6 | 0.49 | 5.0 | 5.3 | 0.42 | 0.04 |
| Overall vaginal maturation | | | | | | | |
| index (% Superficial + | | | | | | | |
| (0.5 × % intermediate) | | | | | | | |
| Placebo | 2.0 | 2.0 | 1.68 | 10.3 | 10.3 | 14.50 | 0.85 |
| DARE-VVA1 1 mg | 4.5 | 15.5 | 23.06 | 18.8 | 18.8 | 26.52 | 0.80 |
| DARE-VVA1 5 mg | 43.5 | 34.6 | 23.90 | 54.3 | 54.3 | 4.60 | 0.55 |
| DARE VVA1 10 mg | 12.5 | 11.5 | 10.04 | 53.0 | 40.5 | 22.52 | 0.54 |
| DARE-VVA1 20 mg | 3.0 | 2.0 | 1.73 | 77.0 | 91.7 | 45.32 | 0.09 |
| DARE VVA1 all actives | 43.5 | 15.9 | 14.68 | 54.3 | 51.30 | 30.4 | 0.32 |
| Superficial cells (%) | | | | | | | |
| Placebo | 0 | 1.0 | 2.00 | 0 | 0 | 0.0 | >0.99 |
| DARSVVA1 1 mg | 4.0 | 2.7 | 2.31 | 1.0 | 1.0 | 1.41 | 0.54 |
| DARE-VVA1 5 mg | 4.0 | 4.3 | 4.20 | 13.5 | 13.5 | 16.26 | 0.82 |
| DAREVVA1 10 mg | 0 | 1.7 | 2.89 | 8.0 | 7.7 | 6.51 | 0.18 |
| DARE-VVA1 20 mg | 0 | 0.0 | 0.00 | 54.0 | 52.3 | 33.53 | 0.06 |
| DARE-VVA1 all actives | 0 | 2.4 | 3.1 | 11 | 20.9 | 27.9 | 0.11 |
| Parabasal cells (%) | | | | | | | |
| Placebo | 96.5 | 97.0 | 2.16 | 79.5 | 79.5 | 28.99 | >0.99 |
| DARE-VVA1 1 mg | 95.0 | 71.7 | 44.81 | 13.5 | 13.5 | 19.09 | 0.39 |
| DARE-VVA1 5 mg | 3.0 | 12.8 | 21.69 | 0.0 | 0 | 0.00 | 0.41 |
| DARE-VVA1 10 mg | 75.0 | 78.7 | 17.79 | 6.0 | 26.7 | 39.31 | 0.19 |
| DARE-VVA1 20 mg | 94.0 | 96.0 | 3.46 | 0.0 | 2.3 | 4.04 | 0.07 |
| DARE-VVA1 all actives | 75.0 | 60.8 | 41.2 | 1.0 | 13.4 | 22.9 | 0.04 |

SD, standard deviation.

### Systemic Plasma Tamoxifen Pharmacokinetic/Local Vaginal Pharmacodynamic Correlations

Plasma tamoxifen concentrations were significantly and negatively correlated with vaginal pH (Spearman R=−0.51, p<0.01) and % vaginal parabasal cells (Spearman R=−0.53, p<0.01). Plasma tamoxifen concentrations were significantly and positively correlated with % vaginal superficial cells (Spearman R=0.45, p<0.01), % vaginal intermediate cells (Spearman R=0.45, p<0.01) and total VMI (Spearman R-0.62, p<0.01).

### Most Bothersome Genitourinary Symptom and Severity of Symptom

Of the 17 enrolled participants, seven reported that vaginal dryness was their MBS and 10 reported that dyspareunia was their MBS at screening. Table 11 presents the severity of the MBS reported by participants in the placebo group versus all active VVA1 users combined for this secondary endpoint. Among active VVA1 users, there was a significant decrease in the severity of vaginal dryness and dyspareunia from baseline at the end of treatment (p=0.02 for both indices). Placebo users did not experience significant changes in the severity of their MBS from baseline (p=0.17 for vaginal dryness and p=0.33 for dyspareunia). Due to sample size, the data were not analyzed by individual active dosing group.

TABLE 11
Frequency of most bothersome genitourinary symptom and severity
by dosing group at pretreatment baseline and end of treatment

| | Number of participants reporting the most | | | | Fisher's |
|---|---|---|---|---|---|
| | bothersome genitourinary symptom and severity | | | | exact |
| Dosing group | None | Mild | Moderate | Severe | p-value |

Vaginal dryness

| | | | | | |
|---|---|---|---|---|---|
| All actives, screening | 0 | 0 | 3 | 2 | 0.02 |
| All actives, end of treatment | 1 | 3 | 0 | 0 | |
| All placebos, screening | 0 | 0 | 1 | 1 | 0.17 |
| All placebos, end of treatment | 1 | 1 | 0 | 0 | |

Pain with intercourse (dyspareunia)

| | | | | | |
|---|---|---|---|---|---|
| All actives, screening | 0 | 0 | 3 | 5 | 0.02 |
| All actives, end of treatment | 4 | 1 | 0 | 2 | |
| All placebos, screening | 0 | 0 | 0 | 2 | 0.33 |
| All placebos, end of treatment | 1 | 0 | 0 | 0 | |

### DISCUSSION

Tamoxifen is a SERM which acts as an estrogen antagonist in the breast; it binds to alpha estrogen receptors in breast tissue, effectively blocking natural estrogen from binding to these sites, and is therefore an effective HR+ breast cancer treatment. Tamoxifen was approved by the US Federal Drug Administration (FDA) in oral dosage forms (tablet and solution) for the prevention and treatment of estrogen receptor-positive breast cancer in 1977. The tissue-specific effects of tamoxifen, importantly, are dependent on the patient's age and endogenous hormonal profile, and currently, oral tamoxifen is primarily given to premenopausal women diagnosed with breast cancer. Oral, systemic tamoxifen is known to cause VVA in premenopausal women by working at the vaginal tissue level to block estrogen activity. Postmenopausal breast cancer patients are often treated with oral aromatase inhibitors, which may amplify VVA by blocking any peripheral conversion of steroid hormones to estradiol (E2) centrally. Thus, the prevalence of VVA in both premenopausal and postmenopausal HR+breast cancer survivors is estimated to be 70%.

A previously conducted, exploratory, proof-of-concept clinical study, where four postmenopausal women with moderate-to-severe WA symptoms were treated with a preliminary formulation of DARE-VVA1 (approximately 13 mg tamoxifen citrate vaginal suppository, produced by a compounding pharmacy) intra-vaginally once daily for a week and then twice weekly for 3 months found decreases in vaginal pH and improvements in patient-reported assessments of vaginal dryness.

This current first-in-woman study of DARE-VVA1 at four dosing strengths (1, 5, 10 and 20 mg) demonstrated that during 8 weeks of use (daily for 2 weeks and twice weekly for 6 weeks), dosed in the same dosing frequency as several other approved micro-dose VVA E2 therapies, DARE-VVA1, in all strengths, was safe and had an AE profile similar to placebo. All TEAEs were mild to moderate, with no participant experiencing a TEAE that was severe or required study drug discontinuation. Vaginal speculum examinations were normal, with most assessments of vaginal edema or erythema graded as not present or mild.

Due to ovulatory concentrations of serum progesterone in premenopausal women, the risk of endometrial hyperplasia with oral tamoxifen use is no different than in the general premenopausal population. However, among postmenopausal women taking daily oral tamoxifen for at least 4 years, the risk of simple hyperplasia is approximately 12%, of complex hyperplasia 3% and of endometrial carcinoma is 2%, which is about two to three times that of the general postmenopausal population. The risk of endometrial hyperplasia was assessed in this first-in-woman study by transvaginal ultrasound measurement of the endometrial stripe width and found that all participants had an endometrial stripe width of ≤4 mm. Although this is reassuring, endometrial tissue biopsies are obtained at pre-treatment baseline and the end of treatment in future studies of DARE-VVA1.

The low-risk systemic safety profile seen in this study was likely due to the minimal systemic drug exposure experienced by active product users, even among women randomized to the highest DARE-VVA1 dose (20 mg). After a single oral dose of tamoxifen (20 mg), the mean maximum plasma concentration of tamoxifen is 40 ng/ml (range 35-45 ng/ml). Mean steady-state concentrations of orally administered tamoxifen (20 mg dose) are 122 ng/ml (range 71-183). Participants in the highest DARE-VVA1 dosing group experienced median plasma maximum observed drug concentration and tamoxifen concentrations that were at least 10-fold less than those seen with systemic, oral tamoxifen use.

Orally dosed tamoxifen is metabolized in the liver by the cytochrome P450 system. The first metabolite of tamoxifen after CYP3A 4/5 degradation is NDT, which is approximately 1.7× more potent systemically than tamoxifen. When dosed orally, the mean peak plasma concentration of NDT is 15 ng/ml (range 10-20 ng/ml). The average steady-state plasma concentration of NDT after 3 months of oral administration of daily 20 mg tamoxifen is 353 ng/ml (range 152-706 ng/ml). During 8 weeks of VVA1 use, the major metabolite of tamoxifen, NDT, was present in plasma at concentrations which were less than 2% of that seen with oral tamoxifen use. NDT is further metabolized into endoxifen by the CYP2D6 enzyme and this enzyme also metabolizes tamoxifen to 4-OHT. Both of these secondary tamoxifen metabolites are approximately 100× more potent than tamoxifen, and were found mostly in the BLQ range for all DARE-VVA1 dosing groups, suggesting minimal impact on systemic exposure. The current PK findings are consistent with the initial proof-of-concept study, which showed median plasma tamoxifen concentrations of 5.8 ng/ml (range 1.0-10.0 ng/ml) measured after 2 months of use with the 13 mg intravaginal dose.

Although the assessment of preliminary efficacy and PD were secondary endpoints, this study demonstrated that topical delivery of tamoxifen resulted in statistically significant decreases in vaginal pH with active product use (all active doses combined), with the 10 mg and 20 mg dosing groups showing the most improvement in vaginal pH over the 8-week treatment. The median end of treatment vaginal pH levels (range 5.0-5.3) remained in what would be considered the menopausal range (≥5.0), which is higher than the vaginal pH levels normally achieved after 4 weeks of micro-dose E2 treatment (<5.0, premenopausal range). This is likely due to the small overall sample size, particularly the fact that only six of the participants who completed the study used either the 10 mg (n=3) or the 20 mg (n=3) dose. Vaginal pH is likely the best marker of vaginal health and because plasma tamoxifen concentrations had a statistically significant inverse relationship with vaginal pH, and plan to move forward with the 10 mg and 20 mg doses.

Similarly, even with micro-dose vaginal therapy, the VMI showed overall improvement with active product use, although the increase in the overall VMI was not statistically significant. The largest increase in total VMI, reflecting a shift to healthy, superficial cells, occurred in the 20 mg DARE-VVA1 dosing group. There was a non-significant increase in superficial cells among active product users, again with the 20 mg dosing cohort experiencing the highest increase in healthy, differentiated superficial cells. However, all active product users had a significant decrease in parabasal cells (p=0.04), with the 20 mg dosing cohort showing the largest decrease in this cell type, which is characteristic of a fragile, atrophic vaginal epithelium. It was found that increasing plasma tamoxifen concentrations were significantly positively correlated with local increases in healthy superficial cells, intermediate cells and overall VMI score. Increasing plasma tamoxifen concentrations were also statistically and inversely correlated with decreasing proportions of parabasal cells. Thus, systemic PK/local PD correlations with VMI data also support forwarding the 10 mg and 20 mg dosing strengths for further testing.

At baseline, participants reported two genitourinary symptoms as being the most bothersome, vaginal dryness and dyspareunia. It is notable that the self-reported severity of both symptoms changed with active product use (p=0.02 for both symptoms), while there was no statistically significant change in the severity of these two symptoms with placebo use. This was a small sample size for this endpoint, but the Fisher exact test was used for these comparisons, which is not dependent on sample size.

This study was limited by the adverse impact of COVID-19 on participant recruitment and thus did not achieve the sample size which was estimated prior to study start. However, both primary endpoints of safety and PK were primarily descriptive in nature, and no discernible safety differences were found between active and placebo product, and the PK data were consistent, with the highest dosing group having the highest systemic tamoxifen and metabolite exposure. Combining all active study doses for comparison of preliminary efficacy endpoints was due to the low sample size and the fact that these endpoints were secondary endpoints and were specified as preliminary efficacy assessments. The preliminary efficacy data complement the PK and safety data and serve to support the selection of the 10 mg and 20 mg doses for further evaluation.

In conclusion, this first-in-woman study of DARE-VVA1 in healthy postmenopausal women demonstrated that DARE-VVA1 was safe, resulted in minimal systemic tamoxifen or tamoxifen metabolite exposure and had TEAEs that were mostly localized to the vagina and were mild or moderate. Although the study was not powered to measure efficacy of the four individual dosing groups, the vaginal pH, vaginal cytology and subjective reports of the severity of MBS support that the 10 mg and 20 mg doses show promise for treating VVA and support continued study of these doses in women with moderate to severe VVA.

### Methods

### Clinical Study

This was a phase 1/2, randomized, double-blind, placebo-controlled study among postmenopausal women with moderate-to-severe VVA at two centers in Australia. The study was approved by the ethics board for each study site (Central Adelaide Local Health Network Human Research Ethics Committee Reference Number 2021/HRE00239) and registered with ClinicalTrials.gov (NCT05378269). Briefly, the sites enrolled healthy women, aged 40-75 years, who were not taking exogenous hormones, and were menopausal as defined by 12 months of spontaneous amenorrhea or 6 months of spontaneous amenorrhea with a serum follicle stimulating hormone (FSH) concentration of 40 mIU/ml or higher. On a 4-point Likert scale of no, mild, moderate or severe symptoms, women had to self-report that at least one of the following symptoms was moderate (level 2) or severe (level 3) in intensity: vaginal dryness; vaginal and/or vulvar irritation/itching; dysuria; or vaginal pain with sexual activity (dyspareunia). Alternatively, women with vaginal bleeding associated with sexual activity were also eligible for the study. Volunteers had to have vaginal cellular cytology with ≤5% superficial cells and vaginal pH>5 at screening. For women with an intact uterus, the endometrial thickness had to be ≤4 mm on transvaginal ultrasound. Subjects had to have a normal mammogram within 2 years of the screening visit, normal cervical cytology cancer screening, a normal baseline 12-lead electrocardiogram and baseline laboratory values either within normal limits or accepted by the investigator and medical monitor as not clinically significant. Participants were excluded if they had significant cardiovascular, renal, pulmonary, neurological or hepatic diseases preventing compliance with the study, if they were currently taking anticoagulant drugs or if they had uncontrolled hypertension. Finally, for this first-in-woman study, women could not have a history of cancer in the past 5 years, undiagnosed vaginal bleeding or a known or suspected estrogen-dependent neoplasia.

Women self-administered a vaginal soft gel capsule once a day for the first 2 weeks and then twice weekly for 6 weeks (approximately 26 doses), which is the normal dosing frequency for many micro-dose vaginal E2 VVA creams and vaginal tablet regimens. They were seen over a total of approximately 10 visits where safety, PK, PD and acceptability endpoints were collected (see Table 12).

TABLE 12
Schedule of Evaluations

| | Screening | | | Administration Period | | | | | | | | | | | | | | FU |
|---|---|---|---|---|---|---|---|---|---|---|---|---|---|---|---|---|---|---|
| | Day(s) | | | | | | | | | | | | | | | | | |
| | −21 to −1 | 1-2 | | | | | 4 | 7 | 10 | 18 | 28 | 42 | 56-57 | | | | | 63 |
| | Time from Admin. (hr.) | | | | | | | | | | | | | | | | | |
| | | Pre-dose | 0 | 4 | 10 | 24 | | | | | | | Pre-dose | 0 | 4 | 10 | 24 | |
| Informed | X | | | | | | | | | | | | | | | | | |
| consent | | | | | | | | | | | | | | | | | | |
| Vaginal | X | X | | | | X | | X | | X | X | X | | | | | X | X |
| speculum exam | | | | | | | | | | | | | | | | | | |
| Vital signs | X | X | | X | X | X | | X | | X | X | X | X | | X | X | X | X |
| 12-Lead ECG | X | X | | X | | | | X | | X | X | | X | | X | | X | X |
| Serum | X | X | | | | | | X | | X | X | | | | | | X | X |
| chemistry, | | | | | | | | | | | | | | | | | | |
| hematology, | | | | | | | | | | | | | | | | | | |
| coagulation | | | | | | | | | | | | | | | | | | |
| panel and | | | | | | | | | | | | | | | | | | |
| urinalysis | | | | | | | | | | | | | | | | | | |
| Vaginal pH | X | X | | | | | | | | X | X | X | | | | | X | X |
| Vaginal | X | X | | | | | | | | X | X | X | | | | | X | X |
| cytology | | | | | | | | | | | | | | | | | | |
| Transvaginal | X | | | | | | | | | | | | | | | | X | |
| ultrasound | | | | | | | | | | | | | | | | | | |
| Assessment of | X | | | | | | | | | X | X | X | X | | | | | X |
| VVA symptoms | | | | | | | | | | | | | | | | | | |
| Diary review | X | X | | | | | X | X | X | X | X | X | X | | | | | |
| and | | | | | | | | | | | | | | | | | | |
| administration | | | | | | | | | | | | | | | | | | |
| Study drug | | | X | | | X | X | X | X | X | X | X | | X | | | | |
| administration | | | | | | | | | | | | | | | | | | |

| | | | | | | | | | | | | | |
|---|---|---|---|---|---|---|---|---|---|---|---|---|---|
| PK blood | X | 0.5, 1, 2, 4, | X | X | X | X | X | X | X | X | 0.5, 1, 2, 4, | X | X |
| samples (trough | | 5, 8, | | | | | | | | | 5, 8, | | |
| unless specified) | | 12 (hr.) | | | | | | | | | 12 (hr.) | | |

### Randomization

In this parallel-group randomized study, women who met the study entry criteria were randomly assigned, in a 1:1:1:1:1 ratio, to one of the five treatment groups (DARE-VVA1 1 mg or 5 mg or 10 mg or 20 mg dose or matching placebo). The randomization schedule was computer-generated using a permuted block algorithm. The study center was not a blocking factor in the randomization schedule. The randomization numbers were assigned sequentially by an un-blinded member of the ICON Clinical Research Team who was not otherwise involved in the study. No one involved in the study performance had access to the randomization schedule before the official un-blinding of treatment assignment.

### Blinding and Un-Blinding Treatment Assignment

The different tamoxifen-dose DARE-VVA1 vaginal capsules and placebo capsules are indistinguishable by appearance, smell and feel. All participants, investigators and study personnel involved in the conduct of the study, including data management, were blinded to treatment assignment with the exception of a specified un-blinded statistician from ICON Clinical Research who had access to the randomization code. The un-blinded study personnel did not participate in study procedures or data analysis prior to un-blinding of the study data to all study-related personnel

### Study Product

DARE-VVA1 was supplied as 1, 5, 10 and 20 mg vaginal capsules or matching placebo capsules by Catalent Pharma Solutions (St. Petersburg, FL, USA). All study products were stored at room temperature.

### Safety Assessments

Safety was assessed primarily through treatment emergent adverse events (TEAEs), which were graded as to their severity and relationship to product use. Any vaginal or vulvar irritation, vaginal discharge or pelvic pain was reported as an adverse event (AE) using standardized Medical Dictionary for Regulatory Activities (MedDRA) codes and graded for severity. Adverse drug reactions (ADRs) were defined as all noxious and unintended responses to a medicinal product, for which a causal relationship between a medicinal product and an AE is at least a reasonable possibility (i.e. the relationship cannot be ruled out). All AEs judged by either the reporting investigator or the sponsor as having a reasonable causal relationship to an investigational product (IP) qualify as ADRs. An unexpected ADR was defined as an ADR for which the nature or severity is not consistent with the applicable product information (e.g. Investigator Brochure for an unapproved IP).

Safety was also measured by changes from screening in clinical laboratory assessments (hematology, serum chemistry, coagulation panel, urinalysis), vital signs, 12-lead electrocardiograms, physical examinations and endometrial stripe width measurements by transvaginal ultrasound. During each vaginal speculum examination, local erythema and edema were graded by the investigator on a 4-point Likert scale of none/absent (score 0), mild (score 1), moderate (score 2) and severe (score 3).

### Pharmacokinetic Evaluations

After baseline evaluations including the time° venous plasma sample, the day 1 DARE-VVA1 dose or matching placebo was self-administered in the clinic and serial peripheral venous samples were drawn from an indwelling line at 0.5, 1, 2, 4, 5, 8, 12 and 24 h. Trough samples were collected 24 h after administration of inserts on days 4, 7, 10, 18, 28, 42 and 56 (end of treatment) and a final sample was collected at follow-up on day 63, approximately 7 days since the last dose. On the last day of dosing (day 56), serial peripheral venous samples were repeated at 0.5, 1, 2, 4, 5, 8, 12 and 24 h.

Plasma concentrations of tamoxifen and three tamoxifen metabolites (N-desmethyltamoxifen [NDT], 4-hydroxyta moxifen [4-OHT] and N-desmethyl-4-hydroxytamoxifen [endoxifen]) were analyzed by Agilex Laboratories using dual tandem liquid chromatography and mass spectrometry (LC/MS-MS). The lower limit of quantitation was 0.1 ng/ml (tamoxifen), 0.1 ng/ml (NDT), 0.05 ng/ml (4-OHT) and 0.05 ng/ml (endoxifen). For PK characterization, concentrations for each dose were calculated using non-compartmental analysis. Concentrations that were below the lower limit of quantitation (BLQ) were estimated as 0.5×lower limit of quantitation. The PK parameter estimates were completed using WinNonlin (Pharsight Corporation). The actual sampling time was used for all parameter estimations. Standard PK parameters assessed included measures of the extent of absorption using estimates of the area under the plasma concentration-time curve, the maximum observed drug concentration and the time to reach the maximum drug concentration.

### Preliminary Pharmacodynamics and Preliminary Treatment Efficacy

Local estrogenic effect measurements included obtaining vaginal epithelial cells from the lateral vaginal sidewall and determining the VMI. The VMI is determined by categorizing the ratio (proportion in 100 cells) of the three types of vaginal epithelial cell types (parabasal, intermediate and superficial). A total VMI score was calculated as: % superficial cells+(0.5 x% intermediate cells)+(0 x % parabasal cells). The VMI and vaginal pH were measured at screening and baseline, and subsequently every 2 weeks during treatment and 1 week after treatment.

At the screening visit, participants rated on a 4-item Likert scale (not present [none], mild, moderate or severe) the severity of the following vaginal symptoms: vaginal dryness; vaginal and/or vulvar irritation/itching; dysuria; and vaginal pain associated with sexual activity (dyspareunia). Additionally, participants indicated whether vaginal bleeding associated with vaginal activity was present or absent. They also selected one of the five symptoms as their MBS. All five symptoms were evaluated again during later study visits and the change in severity from baseline was determined.

### Sample Size and Statistical Analysis

As is the case for first-in-woman safety and PK studies, the primary endpoints of this study were primarily descriptive in nature. The sample size for this first-in-woman safety and PK study was originally estimated to include approximately eight participants in each of the five dosing groups. However, study recruitment was adversely impacted by the COVID-19 pandemic and was halted once approximately half of the enrollment goal was met after a blinded interim analysis of some select secondary endpoints revealed potential treatment efficacy potential.

The following three analysis populations were described: safety population, which included all participants who were enrolled and received any amount of planned IP; intent-to-treat population, which included all randomized participants, who were used for the analysis of secondary efficacy endpoints; and PK population, which included all participants who received at least one dose of IP and provided at least one quantifiable PK plasma sample. Summary statistics were described for each dosing cohort and for the active combined doses (1 mg and 5 mg and 10 mg and 20 mg) versus placebo. For primary safety and PK endpoints, data for each dosing cohort were described separately. Because the sample size for each active dosing cohort was small (n=3-4 participants), comparisons between all active product users versus placebo were used for the secondary endpoints of preliminary product efficacy (vaginal pH, VMI and MBS). Continuous variables included the number of participants, mean, standard deviation, median, minimum and maximum. For categorical variables, the number and percentage of participants in each category is presented. The denominator for the percentage is based on the number of participants appropriate for the purpose of analysis. For all calculations of change from baseline, the last observation recorded before the first administration of the insert (DARE-VVA1 or placebo) was considered as the baseline observation. Paired changes from baseline were compared using a Wilcoxon signed rank sum test, as all continuous variables were not normally distributed. Comparison of categorical variables was done by Fisher's exact test.

Systemic plasma PK/local vaginal PD endpoint correlations were done using the Spearman correlation coefficient. Plasma concentrations of tamoxifen obtained at days 0, 1, 18, 28, 42, 57 and 63 were correlated with vaginal pH measurements and VMI parameters obtained at the same time point for all DARE-VVA1 users combined. p-Values<0.05 were considered statistically significant.

### Example 3: Toxicokinetic Parameters and Safety of Vaginal Tamoxifen Tested Pre-Clinically in Rabbits

Tamoxifen is a selective estrogen receptor modulator (SERM) which has species and tissue specific estrogen agonist and antagonist properties. Given orally in women, tamoxifen has estrogen antagonist activity in breast tissue and estrogen agonist effects in the endometrium and vagina. When administered vaginally in women, there is reduced systemic exposure leading to reduced systemic side effects. The objective of this pre-clinical study was to test the safety, pharmacokinetics (PK) and toxicity of vaginal tamoxifen over a 3 and 9 month treatment period in female New Zealand White rabbits.

### Materials and Methods

All animal studies were conducted under IACUC approvals. In study 3015-012, at Charles River Labs (Mattawan, MI, USA), 45 female rabbits were randomly assigned to either sham (saline), placebo, or 5, 10 or 20 mg/kg/dose of vaginal tamoxifen. Investigational product (IP) was administered intravaginally once daily for 14 days, then twice weekly for up to 10 weeks with necropsy at 12-weeks. In study 858-0032-TX, conducted at WuXi AppTec (Suzhou, Jiangsu, China), 40 female rabbits were randomly assigned to sham (saline), placebo, or 10 or 30 mg/kg/dose of vaginal tamoxifen. IP was administered daily for 14 days and then twice weekly. An interim necropsy was performed at 26-weeks and then at 39-weeks.

### Results

In the 12-week study, tamoxifen-related decreases in the mean weights (absolute and relative to terminal body weight) of the paired ovaries and uterus/cervix were present at 20 mg/kg/day dose. The decreased mean paired ovary weights in the 20 mg/kg/day group correlated with atrophy of interstitial glands microscopically, but the decrease in the mean weight of the uterus/cervix lacked a microscopic correlate. In the 20 mg/kg/day group, cranial and mid sections of vagina, there was minimal to mild diffuse atrophy, characterized by a slight thinning/attenuation of the epithelium. There was an increased incidence and/or severity of minimal to mild mixed inflammatory cell infiltration in the cranial and mid sections of vagina. There was no evidence of epithelial degeneration, erosion, or ulceration in any of the vaginal sections examined at 12-week necropsy. Six rabbits died during the 12-week study of causes not related to the IP.

After 26-weeks of treatment, there were no IP related macroscopic changes in any treatment group. Product-related uterine and ovarian weight decreases were present in the at the 30 mg/kg/day dose, which correlated microscopically with mild to moderate uterine atrophy and moderate to marked atrophy of the interstitial cells in the ovary. At 26-weeks, uterine atrophy was characterized by the overall decrease of uterine horn diameter, with the size ratio of the endometrium to myometrium remaining intact. Four of five females at 30 mg/day were affected and these animals had the lowest uterine weights.

### CONCLUSIONS

In conclusion, once daily intravaginal administration of tamoxifen at 0, 5, 10, and 20 mg/day to rabbits for 12 weeks was tolerated up to the highest dose tested of 20 mg/day. Tamoxifen administration over 12-weeks resulted in non-adverse findings of an increased incidence of ovarian interstitial gland atrophy with decreased weights, decreased uterine weights, diffuse vaginal atrophy, and an increased incidence of vaginal mixed inflammatory cell infiltrates at 20 mg/day. There were no observed tamoxifen-related adverse effects on any of the other parameters. Therefore, the no-observed-adverse-effect level (NOAEL) was 20 mg/day in the 12-week study.

In two studies which dosed vaginal tamoxifen to rabbits for up to 39-weeks, there was no evidence of uterine or endometrial hyperplasia. There were no observed clinical findings which would be suspicious for systemic toxicity. Local genital tract (vagina, uterus, ovary) findings were mostly organ atrophy, with no observed adverse effects at the 20 mg dose over 12-weeks or the 30 mg dose over 39 weeks.

1. A method for treating vulvar and vaginal atrophy (VVA) or one or more symptoms in a subject in need thereof, comprising administering to a vagina a composition comprising a vaginal insert and tamoxifen.

2. The method of claim 1, wherein the vaginal insert is a soft gelatin capsule-based vaginal insert.

3. The method of claim 1, wherein the composition comprises a salt of tamoxifen.

4. The method of claim 1, wherein the composition comprises a citrate salt of tamoxifen.

5. The method of claim 1, wherein the composition comprises at least about 1 mg to about 20 mg tamoxifen.

6-8. (canceled)

9. The method of claim 1, wherein the one or more symptoms are selected from vaginal dryness, vaginal and/or vulvar irritation, vaginal and/or vulvar itching, dysuria, vaginal pain with sexual activity (dyspareunia), and/or vaginal bleeding associated with sexual activity.

10. The method of claim 1, wherein the one or more symptoms comprises a serum level of Folicle-Stimulating Hormone (FSH) greater than 40 mIU/ml.

11. The method of claim 1, wherein the one or more symptoms comprises a vaginal pH greater than 5.

12. The method of claim 1, wherein the one or more symptoms comprises less than 5 percent superficial cells on a vaginal smear.

13. The method of claim 1, wherein the subject is a postmenopausal woman with a uterus or a postmenopausal woman without a uterus.

14. The method of claim 1, wherein the subject is a hormone adverse woman.

15. The method of claim 1, wherein the subject is a woman with current breast cancer, a history of breast cancer, or at an increased risk of breast cancer.

16. The method of claim 1, wherein the subject is a woman with current or a history of hormone receptor-positive (HR+) breast cancer.

17. The method of claim 1, wherein the subject is a woman with a history of breast cancer or a mammogram that is positive or suspect for breast cancer or breast cancer occurring in an identical twin.

18. The method of claim 1, wherein the subject is a woman with a history of thrombophilia.

19. The method of claim 1, wherein the composition is administered to a vagina by self-placement.

20. The method of claim 1, wherein the composition is administered one dose per day for 14 days followed by two doses per week for 6 weeks.

21. The method of claim 1, wherein the method comprises measurement of serum levels of 4-hydroxytamoxifen, N-desmethyltamoxifen, and/or N-desmethyl-4-hydroxytamoxifen.

22. The method of claim 1, wherein the method comprises measurement of vaginal pH.

23. The method of claim 1, wherein the method comprises measurement Vaginal Maturation Index (VMI).

24. A pharmaceutical composition comprising a vaginal insert and tamoxifen.

25. The pharmaceutical composition of claim 24, wherein the vaginal insert is a soft gelatin capsule-based vaginal insert.

26. The pharmaceutical composition of claim 24, wherein the composition comprises at least about 1 mg to about 20 mg tamoxifen.

27-29. (canceled)

30. A kit comprising the pharmaceutical composition of claim 24 and a dispenser for the composition.

31. The kit of claim 30, wherein the dispenser contains a single unit dosage form of the composition.

32. The kit of claim 30, wherein a single unit dosage comprises between about 1 mg and about 20 mg of the composition.

33. (canceled)
